(19) United States
(12) Patent Application Publication
(10) Pub. No.: US 2005/0008618 A1
(43) Pub. Date: Jan. 13, 2005

(54) Composition for delivering an agent to a target cell and uses thereof

(76) Inventors: Howard Kaufman, New York, NY (US); Michal Bereta, Krakow (PL)

Correspondence Address: Leslie Gladstone Restaino, Esq.;Brown Raysman Millstein Felder & Steiner LLP, 163 Madison Avenue, P.O. Box 1989, Morristown, NJ, 07962-1989, US

(21) Appl. No.: 10/789,627

(22) Filed: Feb. 26, 2004

Related U.S. Application Data
(60) Provisional application No. 60/450,719, filed on Feb. 27, 2003.

(51) Int. Cl. A61K 48/00; C12N 1/21; C12N 1/18

(52) U.S. Cl. 424/93.2; 435/252.3; 435/254.2

(57) ABSTRACT

The present invention provides a composition for delivering an agent to a target cell, comprising: (a) a microorganism that has, on its cell surface, at least one exogenous molecule that binds to an antigen on the surface of a target cell; and (b) an agent. The present invention further provides a vaccine comprising: (a) at least one microorganism that has, on its cell surface, at least one exogenous molecule that binds to an antigen on the surface of a target cell; (b) an agent; and (c) a pharmaceutically-acceptable carrier. Also provided are methods for treating and preventing neoplasia in a subject in need of treatment, by administering to the subject a composition or a vaccine of the present invention.

### CROSS-REFERENCE TO RELATED APPLICATIONS

This application claims the benefit of U.S. Ser. No. 60/450,719 filed Feb. 27, 2003.

### BACKGROUND OF THE INVENTION

Neoplasia is a disease characterized by an abnormal proliferation of cells known as a neoplasm. Neoplasms may manifest in the form of a leukemia or a solid tumor, and may be benign or malignant. Malignant neoplasms, in particular, can result in a serious disease state, which may threaten life. Significant research efforts and resources have been directed toward the elucidation of anti-neoplastic measures, including chemotherapeutic agents, which are effective in treating patients suffering from neoplasia. Effective anti-neoplastic agents include those which inhibit or control the rapid proliferation of cells associated with neoplasms, those which effect regression or remission of neoplasms, and those which generally prolong the survival of patients suffering from neoplasia. Successful treatment of malignant neoplasia, or cancer, requires elimination of all malignant cells, whether they are found at the primary site, or have extended to local/regional areas, or have metastasized to other regions of the body. The major therapies for treating neoplasia are surgery and radiotherapy (for local and local/regional neoplasms) and chemotherapy (for systemic sites) (Beers and Berkow (eds.), The Merck Manual of Diagnosis and Therapy, 17^{th} ed. (Whitehouse Station, N.J.: Merck Research Laboratories, 1999) 973-74, 976, 986, 988, 991).

Despite the various methods for diagnosing and treating cancers, the disease remains prevalent in all segments of society, and is often fatal. Clearly, alternative strategies for detection and treatment are needed to improve survival in cancer patients. In particular, improved methods for achieving targeted delivery of therapeutic compounds to the sites of solid-tumor growth would provide a strong basis from which novel cancer-treatment regimens may be developed.

A variety of biological delivery systems (e.g., antibodies, bacteria, liposomes, and viruses) currently exist for delivering cytotoxic drugs, genes, immunostimulators, pro-drug converting enzymes, radiochemicals, and other therapeutic agents to the vicinity of solid tumors or neoplastic cells (see, e.g., Ng et al., An anti-transferrin receptor-avidin fusion protein exhibits both strong proapoptotic activity and the ability to deliver various molecules into cancer cells. Proc. Natl. Acad. Sci. USA, 99:10706-11, 2002; Mastrobattista et al., Functional characterization of an endosome-disruptive peptide and its application in cytosolic delivery of immunoliposome-entrapped proteins. J. Biol. Chem., 277:27135-43, 2002; Fefer, “Special delivery” to cancer cells. Blood, 99:1503-04, 2002; Kwong et al., The suppression of colon cancer cell growth in nude mice by targeting β-catenin/TCF pathway. Oncogene, 21:8340-46, 2002; Huser et al., Incorporation of decay-accelerating factor into the baculovirus envelope generates complement-resistant gene transfer vectors. Nat. Biotechnol., 19:451-55, 2001; Lu et al., Polymerizable Fab′ antibody fragments for targeting of anticancer drugs. Nat. Biotechnol., 17:1101-04, 1999; Chu et al., Toward highly efficient cell-type-specific gene transfer with retroviral vectors displaying single-chain antibodies. J. Virol., 71:720-25, 1997). For example, U.S. Pat. No. 6,491,905 provides a prokaryotic cell stably carrying a vector that includes a DNA sequence encoding a purine nucleotide phosphorylase or hydrolase, and the use of such a cell, together with a purine pro-drug, to treat tumors. Such treatment options, however, are frequently incapable of accomplishing targeted delivery of therapeutic agents in concentrations sufficient to eradicate the neoplasm, while, at the same time, minimizing damage to surrounding normal tissue.

Apart from their use as drug-delivery vehicles, bacteria and other microorganisms may have therapeutic value as parasites that infect neoplastic cells and inhibit their proliferation. For more than two hundred years, in fact, it has been known that neoplasms may regress, or completely disappear, following acute bacterial infections (see, e.g., Nauts et al., A review of the influence of bacterial infection and of bacterial products (Coley's toxins) on malignant tumors in man. Acta Medica. Scandinavica, 145(Suppl. 276): 1-102, 1953). U.S. Pat. No. 6,190,657, for example, discloses the screening and isolation of super-infective, tumor-specific parasite vectors, such as Salmonella typhimurium and Mycobacterium avium. The vectors are engineered to carry “suicide genes” and/or gene products to the vicinity of the tumor cells. Methods of treatment of solid tumors using these vectors are also disclosed. Similarly, U.S. Pat. No. 6,447,784 discloses a means for enhancing the safety of tumor-targeted bacteria, e.g., by genetic modification of the lipid A molecule. The anti-tumor effects of the attenuated tumor-targeted bacteria may be further enhanced by the expression of pro-drug converting enzymes, such as Herpes simplex virus thymidine kinase (TK), cytosine deaminase (CD), and p450 oxidoreductase.

Despite advantages offered by these parasite-based treatment approaches, their applications are severely limited by a number of factors. For example, a particular strain of microorganism is only capable of infecting certain types of neoplastic cells, since it arises by natural selection. Therefore, the number of neoplastic disorders that may be potentially treated by the particular microorganism is greatly limited.

In view of the foregoing, it is clear that there are limitations to the use of microorganisms as drug-delivery systems and as parasite-based cancer therapies. Accordingly, there exists a need in the art to provide drug-delivery systems which are capable of targeting specific neoplastic cells and which also have the ability to infect a wide variety of neoplastic cells.

### SUMMARY OF THE INVENTION

The inventors describe herein a means for improving, and enhancing the safety of, bacterial vectors that are used to deliver genes, drugs, and other therapeutic compounds into specific tumor cells. More particularly, the invention is directed to a bacterium (attenuated Salmonella typhimurium, strain VNP20009) that has been genetically modified, using plasmid technology, so that it transiently expresses, and displays on the bacterial surface, an antibody (a single-chain variable fragment (scFv)) specific for a tumor antigen (carcinoembryonic antigen (CEA), a membrane-bound glycoprotein expressed abundantly on epithelial cancerous cells). Adhesion of a bacterium to its target cell is the first step required for infection, and CEA is a target for bacterial adhesion and subsequent infection. Thus, display of high-affinity, CEA-specific scFv on the surface of bacterial carriers can ensure highly-specific cargo delivery into disease-affected cells that express the appropriate cell-surface ligand. This invention is an improvement over prior art, in that it combines highly-specific recognition of cell-surface molecules by monoclonal antibodies with the great capacity of bacteria for storing and carrying genetic information.

The bacterial vector of the present invention may be used selectively to deliver genes and other drugs to CEA-expressing cells. The general approach also may be used to target other cell-surface receptors or molecules, thereby providing a technique for highly-selective gene delivery to individual cells. Such an approach may be useful for gene-therapy strategies to treat cancer, genetic diseases, infectious diseases, and other human conditions where gene replacement or expression is indicated. This invention will help overcome one of the major problems in the chemotherapy and immunotherapy of solid tumors: delivery of therapeutic agents into tumor cells, or focusing of immune responses on neoplastic tissue, while simultaneously minimizing damage to normal cells.

Accordingly, the present invention provides a composition for delivering an agent to a target cell, comprising: (a) a microorganism that has, on its cell surface, at least one exogenous molecule that binds to an antigen on the surface of a target cell; and (b) an agent.

The present invention further provides a vaccine comprising: (a) at least one microorganism that has, on its cell surface, at least one exogenous molecule that binds to an antigen on the surface of a target cell; (b) an agent; and (c) a pharrnaceutically-acceptable carrier.

Additionally, the present invention provides a method for treating neoplasia in a subject in need of treatment, by administering to the subject a therapeutic composition in an amount effective to treat the neoplasia, wherein the therapeutic composition comprises: (a) a microorganism that has, on its cell surface, at least one exogenous molecule that binds to an antigen on the surface of a neoplastic cell in the subject; and (b) a therapeutic agent.

Also provided is a method for preventing neoplasia in a subject in need of prevention, comprising administering to the subject a preventive composition in an amount effective to prevent the neoplasia, wherein the preventive composition comprises: (a) a microorganism that has, on its cell surface, at least one exogenous molecule that binds to an antigen on the surface of a neoplastic cell in the subject; and (b) a preventive agent.

The present invention further provides a method for treating neoplasia in a subject in need of treatment, by administering to the subject a therapeutic composition in an amount effective to treat the neoplasia, wherein the therapeutic composition consists of a microorganism that has, on its cell surface, at least one exogenous molecule that binds to an antigen on the surface of a neoplastic cell in the subject.

Finally, the present invention provides a method for preventing neoplasia in a subject in need of prevention, by administering to the subject a preventive composition in an amount effective to prevent the neoplasia, wherein the preventive composition consists of a microorganism that has, on its cell surface, at least one exogenous molecule that binds to an antigen on the surface of a neoplastic cell in the subject.

Additional aspects of the present invention will be apparent in view of the description which follows.

### BRIEF DESCRIPTION OF THE FIGURES

FIG. 1 sets forth a schematic diagram of the plasmid used for expression of a targeting agent (a CEA-specific single-chain antibody fragment) and a therapeutic protein in a Salmonella vector. MoPac2-scFv-ther (Pan-Ther) is a 6-kb plasmid shown with two expression cassettes: (1) the lac promoter (Plac), which is repressed by lacI^{q}, and controls prokaryotic expression of a tripartite fusion protein (Lpp-OmpA-scFv) that is upregulated upon induction with isopropylthio-β-D-galactoside (IPTG); and (2) the cassette for expression of a therapeutic protein of interest (ther.gene). A strong CMV IE promoter (PCMV) regulates expression of the therapeutic protein in eukaryotic cells. A multiple cloning site is located directly downstream from the PCMV transcriptional start site, which allows for easy cloning of any gene of interest. A polyadenylation signal (polyA) is included to ensure proper processing in eukaryotic cells. Chloramphenicol acetyltransferase (CmR) facilitates selection of the plasmid-carrying colonies in the presence of chloramphenicol. Since several Salmonella strains (but not VNP20009 or SL7207) carry natural resistance to chloramphenicol, this resistance gene was chosen to limit antibiotic resistance of the strain in respect of in vivo applications. ColE1 is an origin of replication that allows for maintenance of high copy numbers of the plasmid (more than 100 per cell). Several stretches of the plasmid, particularly fusion sites, have been sequenced.

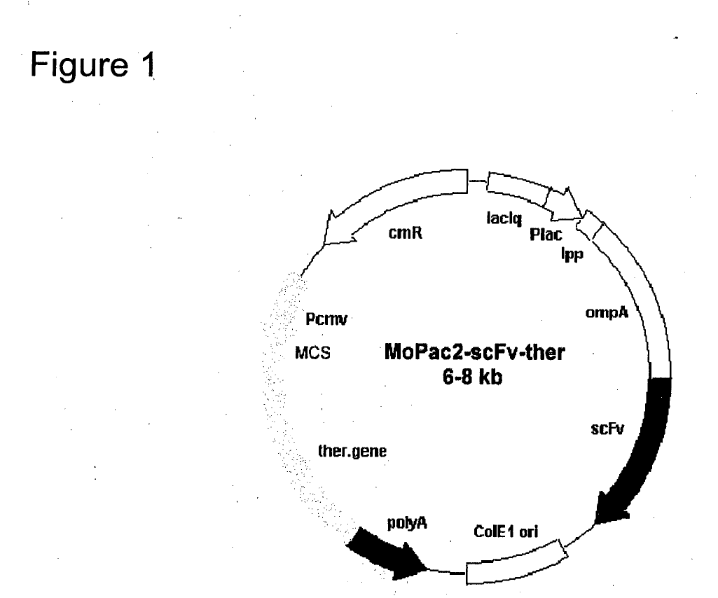

FIG. 2. depicts a Western-blot analysis of Lpp-OmpA-scFv fusion proteins in Salmonella typhimurium VNP20009. Bacteria were transformed by electroporation with Pan-Ther plasmids in which scFv contained either 8 aa (right side of the panel) or 18 aa (left side of the panel) linkers between VL and VH sequences. Samples from overnight cultures of bacteria grown in Terrific broth (TB) at 25° C., in the absence (IPTG 0) or in the presence of inducing agent (IPTG 50-400 μM), were normalized by spectrophotometric measurement, to ensure equal loading, and lysed directly in loading buffer. Electrophoresis in SDS-polyacrylamide gel (10%) and electroblotting were performed according to standard procedures (Sambrook et al., Molecular Cloning: A Laboratory Manual, 2^{nd} ed. (Cold Spring Harbor, N.Y.: Cold Spring Harbor Laboratory Press, 1989)). After blocking with dry milk, nitrocellulose membranes were incubated with goat-anti-mouse primary antibody, followed by HRP-conjugated donkey-anti-goat antibodies, and bands were visualized using chemiluminescence (ECL) technology. A band at approximately 42 kD (Rainbow standards were used for sizing) corresponds to non-degraded Lpp-OmpA-scFv fusion protein. Bands in the absence of IPTG indicate poor control of lacI^{q} repressor over lac promoter, resulting in limited inducibility of the protein (about 20-fold at the optimal concentration of IPTG). Similar results were obtained from analysis of Lpp-OmpA-scFv expression in the strain, SL7207.

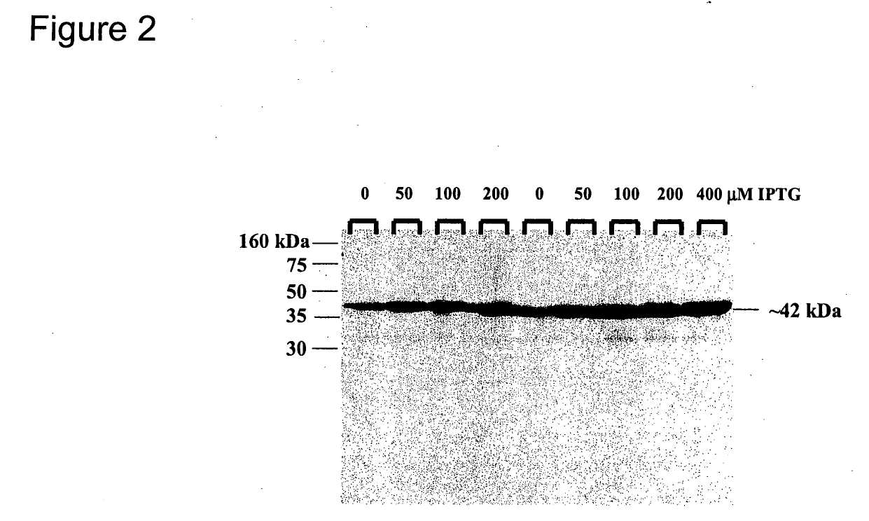

FIG. 3. presents flow cytometric analysis of scFv/L18 and scFv/L8 on the surface of Salmonella typhimurium VNP20009. Bacteria from overnight cultures (about 75% viability) were washed extensively with staining buffer (PBS with 1% BSA and 0.02% sodium azide), and incubated with Ig-specific FITC-conjugated goat-anti-mouse F(ab)₂ fragments. After washing, samples were analyzed by FACSCalibur. Gates were set on live bacteria that showed more than 50% (at 1:10 dilution of anti-mouse Ab from Zymed Laboratories, San Francisco, Cailf.) of highly-positive staining (IPTG 200 μM), and a corresponding lower percentage of positive staining at lower concentrations of IPTG. Control FITC-conjugated goat-anti-rabbit F(ab)₂ fragments did not stain bacteria at any dose of IPTG. Despite substantial Plac “leakage” observed in the Western-blot analysis, less than 2% of bacteria displayed scFv on the surface. ScFv/L18 showed consistently higher surface expression than did scFv/L8. VNP20009 consistently expressed more (about 3 fold) scFv than did SL7207. This demonstrates that the scFv/L18 is optimal for diabody expression on the surface of Salmonella.

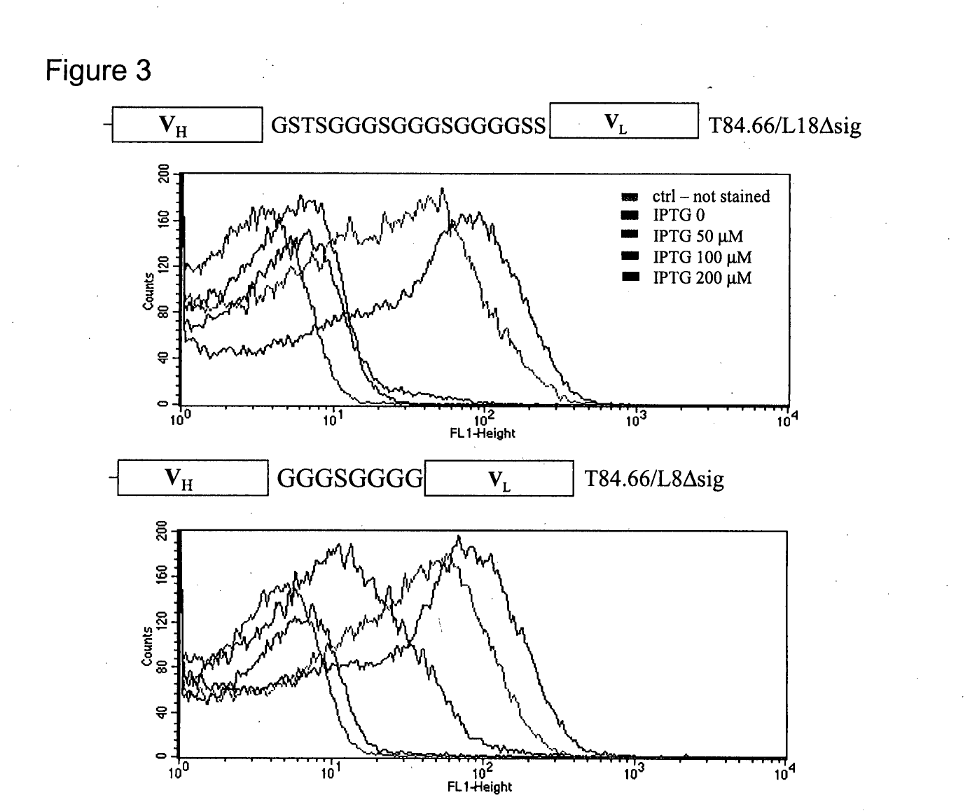

FIG. 4 illustrates binding to antigen by scFv expressed on the surface of Salmonella typhimurium VNP20009. In order to confirm that the CEA-specific scFv molecules were folded properly and functional (i.e., could bind the antigen), CEA was conjugated with FITC (1:3 molar ratio), and used to visualize scFv-CEA complexes on the bacterial surface. After washing, bacteria from overnight cultures were incubated on ice, for 30 min, with CEA-FITC or FITC-conjugated goat-anti-mouse F(ab)₂ fragments. BSA-FITC was used as an additional control. Left panels show staining of VNP20009 expressing non-induced scFv/8L (left upper panel) or scFv/18L (left lower panel). Right panels show staining of IPTG- (200 μM) induced scFv/8L (upper right panel) or scFv/18L (lower right panel). BSA-FITC did not show any binding to non-induced or induced scFv (data not shown). ScFv/L18 on the surface of VNP20009 binds consistently more CEA than scFv/L8. Consequently, scFv/L18 was chosen for use in further experiments.

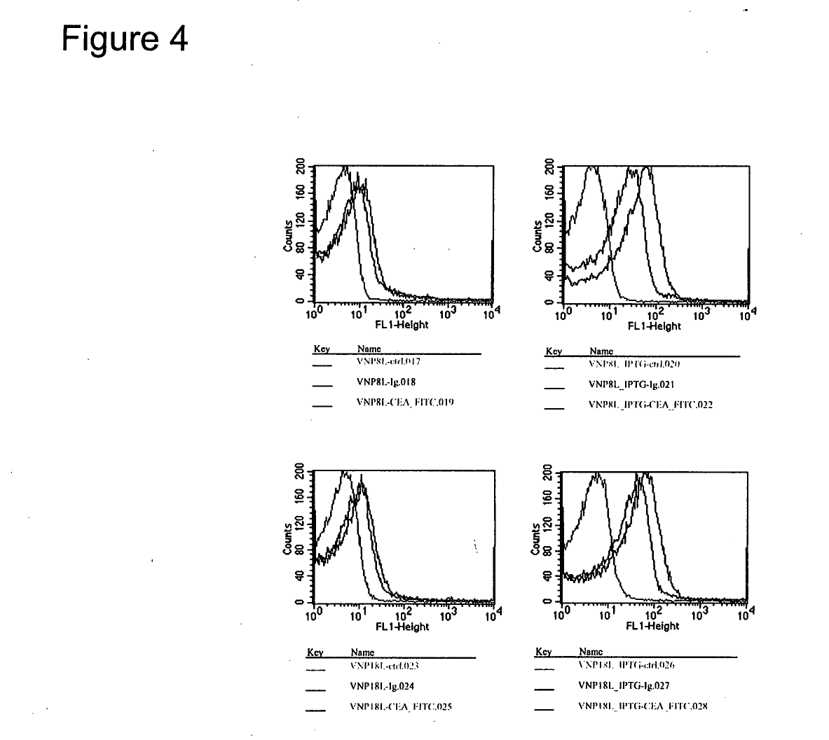

FIG. 5 depicts competitive inhibition of CEA-FITC binding to scFv/18L on the surface of VNP20009 by non-labeled CEA. To confirm specificity of antigen-antibody interaction, highly-purified non-labeled CEA was used to compete with binding of CEA-FITC to scFv/L18 on the surface of Salmonella. After washing, bacteria from overnight cultures were incubated on ice for 30 min with CEA or with control proteins, followed by an additional 30 min of incubation with CEA-FITC. As shown, binding of CEA-FITC was inhibited by about 50% in the presence of equimolar amounts (1 mg/mL) of non-labelled CEA. Non-labelled BSA, as well as non-labelled anti-rabbit Ig, at the same concentrations as CEA, had no effect on the levels of CEA-FITC binding (data not shown). These results confirm the specificity of interaction between bacterial-expressed scFv and CEA.

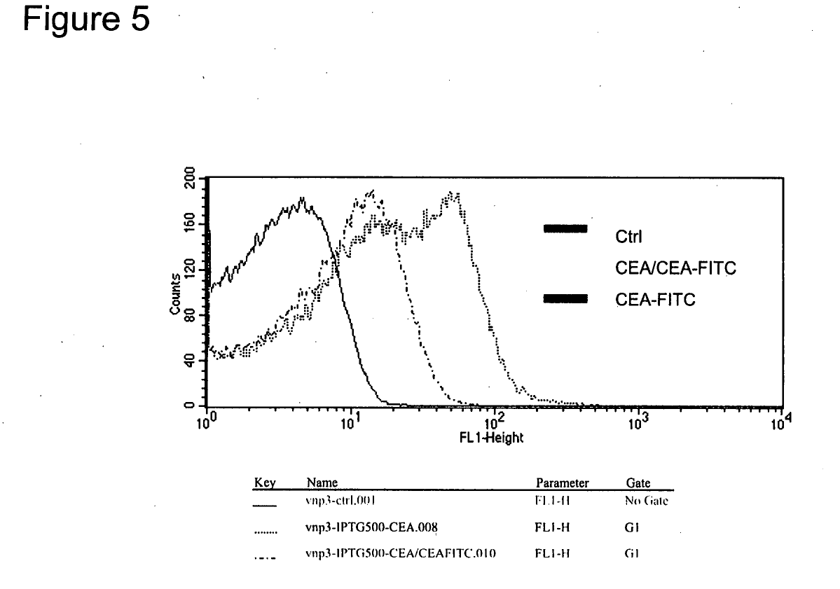

### DETAILED DESCRIPTION OF THE INVENTION

The present invention provides a composition for delivering an agent (e.g., a diagnostic agent, a labelling agent, a preventive agent, or a therapeutic agent, including nucleic acids and polypeptides) to and/or into a target cell, either in vitro or in vivo. The agent is delivered to the target cell by a microorganism, particularly a recombinantly-engineered microorganism. The composition of the present invention comprises: (a) a microorganism that has, on its cell surface, at least one exogenous molecule that binds to an antigen on the surface of a target cell; and (b) an agent. The microorganism of the composition is directed to the target cell, and is capable of delivering its cargo (the agent) thereto, because the exogenous molecule that the microorganism has on its cell surface recognizes an antigen on the surface of the target cell.

In the composition of the present invention, the microorganism may be any alga, bacterium, fungus (including yeast), protozoan, or other microorganism. In a preferred embodiment of the present invention, the microorganism is a bacterium. Examples of bacteria for use in the present invention include, without limitation, Bordetella spp., Borrelia burgdorferi, Brucella melitensis, Chlamydia trachomatis, Clostridium spp., Eimeria acervulina, Encephatozoon cuniculi, Escherichia coli, Legionella pneumophila, Leptomonas karyophilus, Listeria monocytogenes, Mycobacterium avium, Mycobacterium bovis, Mycobacterium tuberculosis, Mycoplasma hominis, Neospora caninum, Nosema helminthorum, Phytomonas spp., Rickettsiae quintana, Salmonella spp., Sarcocystis suihominis, Shigella spp., Streptococcus spp., Treponema pallidum, Yersinia enterocolitica, and Unikaryon legeri. In a preferred embodiment, the bacterium is Escherichia coli, a Mycobacterium spp., a Salmonella spp., or a Shigella spp. More preferably, the bacterium is a Salmonella spp. Examples of Salmonella bacteria for use in the present invention include, without limitation, Salmonella arizonae, Salmonella choleraesuis, Salmonella enteritidis, Salmonella typhi, and Salmonella typhimurium. Serotypes of Salmonella are also encompassed herein. Preferably, the Salmonella bacterium is Salmonella typhimurium. More preferably, the Salmonella bacterium is Salmonella typhimurium VNP20009 or SL7207.

Other microorganisms that may be useful for the purposes of the present invention include, without limitation, the fungi Aspergillus fumigatus, Blastomyces dermatitidis, Candida albicans, Coccidioides immitis, Cryptococcus neoformans, Histoplasma capsulatum, Pneumocystis carinii, and Pythium insidiosum, and the protozoa Acanthamoeba spp., Cryptosporidium spp., Entamoeba histolytica, Giardia lamblia, Leishmania amazonensis, Leishmania major, Leishmania mexicana, Leishmania tropia, Trypanasoma cruzi, Toxoplasma gondii, and Prototheca wickerhamii.

In one embodiment of the present invention, the microorganism functions under both aerobic and anaerobic conditions. In another embodiment of the present invention, the microorganism expresses the exogenous molecule (e.g., when the molecule is a protein). In a preferred embodiment, the microorganism transiently expresses the exogenous molecule.

In another preferred embodiment of the present invention, the microorganism is attenuated. Many of the microorganisms encompassed by the present invention are known to be pathogens in humans and animals. For example, both Gram-negative and Gram-positive bacteria may cause severe (often fatal) septic shocks in humans and animals, by producing endotoxins and autotoxins (Opal et al., Endotoxin as a drug target. Crit. Care Med., 31:S57-64, 2003; Friedman et al., Has the mortality of septic shock changed with time. Crit. Care Med., 26:2078-86, 1998; Bone, R. C., Gram-negative sepsis. Background, clinical features, and intervention. Chest, 100:802-08, 1991). Therefore, to ensure the safe use of the microorganisms of the present invention, and the compositions in which they are found (which may be administered to humans and animals), it is preferred that the virulence of these microorganisms be attenuated.

As used herein, the term “attenuated” refers to a strain of a pathogenic (disease-causing) microorganism that cannot cause serious disease in a host. The end result of attenuation is that the risk of toxicity (and other side-effects) is decreased when the microorganism is administered to, or infects, a host. In a preferred embodiment of the present invention, the microorganism is an attenuated bacterium. In another preferred embodiment of the present invention, the attenuated bacterium is an attenuated Salmonella. More preferably, the bacterium is an attenuated Salmonella typhimurium VNP20009 or SL7207.

To achieve attenuation, the microorganism of the present invention may be modified so that it is less pathogenic. Additionally, the microorganism, or a vector comprising same, may be modified so that a lower titer of that microorganism or vector, when administered to a host, will still achieve results comparable to those obtained with administration of a higher titer of the parental microorganism or vector. Methods for attenuating a microorganism, so as to reduce the risk of potentially-harmful effects to normal cells in the host, include, without limitation, mutagenesis of the microorganism; isolation of microorganism mutants with reduced ability to infect normal host cells in the host's body; isolation of mutants with a genetically-altered lipopolysaccharide composition; and isolation of mutants with altered virulence genes. Other techniques for producing attenuated microorganisms include, without limitation, screening and isolation of naturally-existing attenuated microorganisms (e.g., the isolation of antibiotic-sensitive strains of microorganisms; the isolation of strains that lack virulence factors required for survival in normal cells, especially macrophages and neutrophils; and the isolation of strains of microorganisms with altered cell-wall lipopolysaccharides).

For the purposes of the present invention, it is desirable to produce the attenuated microorganisms using genetic engineering. Standard molecular-biology techniques may be used for such purposes, including, without limitation, gene knockout, random or targeted mutagenesis by chemical or transposon mutagenesis, PCR-based mutagenesis, and gene-silencing using antisense technology or interference RNA (“RNAi”). In a preferred embodiment of the present invention, nucleic acid sequences that encode for the virulence factors in the microorganisms—which factors are essential for survival of the microorganisms in the host cells (especially macrophages and neutrophils)—are deleted, disrupted, or silenced.

A number of virulence factors have been identified in Salmonella. Many, but not all, of these studied virulence factors are associated with survival in macrophages. For example, these factors may be specifically expressed within macrophages, or used to induce specific host cell responses, e.g., macropinocytosis (Fields et al., Mutants of Salmonella typhimurium that cannot survive within the macrophage are avirulent. Proc. Natl. Acad. Sci. USA, 83:5189-93, 1986). Examples of Salmonella virulence factors, which result in attenuated Salmonella if deleted, disrupted, or silenced, include, but are not limited to, the following: cytolysin, DnaK, GroEL, lipid A acyltransferase, long polar fimbriae proteins (such as the protein products of the lpfA, lpfB, lpfC, lpfD, and IpfE genes), 5′-phosphoribosyl-5-aminoimidazole synthetase, and porin proteins (such as the protein products of the envZ, ompC, ompD, ompF, ompR, scrY, scrK, and tppB genes). See, e.g., Oscarsson et al., Characterization of a pore-forming cytotoxin expressed by Salmonella enterica serovars typhi and paratyphi A. Infect. Immun., 70:5759-69, 2002; Mei et al., Optimization of tumor-targeted gene delivery by engineered attenuated Salmonella typhimurium. Anticancer Res., 22:3261-66, 2002; Bang et al., OmpR regulates the stationary-phase acid tolerance response of Salmonella enterica serovar typhimurium. J. Bacteriol., 182:2245-52, 2000; Clairmont et al., Biodistribution and genetic stability of the novel antitumor agent VNP20009, a genetically modified strain of Salmonella typhimurium. J. Infect. Dis., 181:1996-2002, 2000; Norris et al., Phase variation of the lpf operon is a mechanism to evade cross-immunity between Salmonella serotypes. Proc. Natl. Acad. Sci. USA, 96:13393-398, 1999; Low et al., Lipid A mutant Salmonella with suppressed virulence and TNFα induction retain tumor-targeting in vivo. Nat. Biotechnol., 17:37-41, 1999; Mills et al., Trafficking of porin-deficient Salmonella typhimurium mutants inside HeLa cells: ompR and envZ mutants are defective for the formation of Salmonella-induced filaments. Infect. Immun., 66:1806-11, 1998; Khan et al., A lethal role for lipid A in Salmonella infections. Mol. Microbiol., 29:571-79, 1998; Tang et al., Induction and characterization of heat shock proteins of Salmonella typhi and their reactivity with sera from patients with typhoid fever. Infect. Immun., 65:2983-86, 1997; Baumler et al., The lpf fimbrial operon mediates adhesion of Salmonella typhimurium to murine Peyer's patches. Proc. Natl. Acad. Sci. USA, 93:279-83, 1996; Baumler et al., Identification and sequence analysis of IpfABCDE, a putative fimbrial operon of Salmonella typhimurium. J. Bacteriol., 177:2087-97, 1995; and Buchmeier et al., Induction of Salmonella stress proteins upon infection of macrophages. Science, 248:730-32, 1990.

Microorganisms for use in the present invention may also be attenuated by modifying those molecules in the microorganisms that are responsible for their pathological activities. For example, lipopolysaccharides (LPSs) and endotoxins are primarily responsible for causing bacterial sepsis in host organisms. The component of LPS which results in this pathological reaction is lipid A (LA) (Khan et al., A lethal role for lipid A in Salmonella infections. Mol. Microbiol., 29:571-79, 1998). Elimination or reduction of the toxic effects of LA decreases the virulence of the microorganism, resulting in an attenuated microorganism, because the risk of septic shock in the host is reduced, and, therefore, higher levels of the microorganism can be tolerated. See, e.g., Bentala et al., Removal of phosphate from lipid A as a strategy to detoxify lipopolysaccharide. Shock, 18:561-66, 2002; Johnson et al., Structural characterization of monophosphoryl lipid A homologs obtained from Salmonella minnesota Re595 lipopolysaccharide. J. Biol. Chem., 265:8108-16, 1990; and Kanegasaki et al., Structure-activity relationship of lipid A: comparison of biological activities of natural and synthetic lipid A's with different fatty acid compositions. J. Biochem. (Tokyo), 99:1203-10, 1986.

Additionally, the LPS of a bacterium (e.g., Salmonella) may be modified by introducing mutations into the LPS biosynthetic pathway. Several key enzymatic steps in LPS biosynthesis, and the genetic loci controlling them, have been identified in a number of microorganisms (e.g., Campylobacter coli, Campylobacter jejuni, E. coli, Pasteurella haemolytica, Pseudomonas aeruginosa, and Salmonella typhimurium). See, e.g., Belanger et al., Functional analysis of genes responsible for the synthesis of the B-band O antigen of Pseudomonas aeruginosa serotype O6 lipopolysaccharide. Microbiology, 145:3505-21, 1999; Khan et al., A lethal role for lipid A in Salmonella infections. Mol. Microbiol., 29:571-79, 1998; Klena et al., Cloning, sequencing, and characterization of the lipopolysaccharide biosynthetic enzyme heptosyltransferase I gene (waaC) from Campylobacter jejuni and Campylobacter coli. Gene, 222:177-85, 1998; Allen et al., The identification, cloning and mutagenesis of a genetic locus required for lipopolysaccharide biosynthesis in Bordetella pertussis. Mol. Microbiol., 19:37-52, 1996; Potter et al., Cloning and characterization of the galE locus of Pasteurella haemolytica A1. Infect. Immun., 64:855-60, 1996; Marolda et al., Identification, expression, and DNA sequence of the GDP-mannose biosynthesis genes encoded by the O7 rfb gene cluster of strain VW187 (Escherichia coli O7:K1). J. Bacteriol., 175:148-58, 1993; and Raetz, Bacterial endotoxins: extraordinary lipids that activate eucaryotic signal transduction. J. Bacteriol., 175:5745-53, 1993.

A number of mutant strains of Salmonella typhimurium and E. coli, with genetic and enzymatic lesions in their LPS pathways, have been isolated. One such mutant, firA, has a mutation within the gene that encodes the enzyme, UDP-3-O-(R-3-hydroxymyristoyl)-glycocyamine N-acyltransferase, which regulates the third step in endotoxin biosynthesis (Kelly et al., The firA gene of Escherichia coli encodes UDP-3-O-(R-3-hydroxymyristoyl)-glucosamine N-acyltransferase. The third step of endotoxin biosynthesis. J. Biol. Chem., 268:19866-74, 1993). ThefirA mutants of both E. coli and S. typhimurium have a decreased LPS synthesis rate. The mutants produce a lipid A that contains a seventh fatty acid, a hexadecanoic acid. Experimental analysis of the enzymatic activity of other enzymes involved in lipid A biosynthesis has revealed that the firA mutations pleiotropically affect LPS biosynthesis. The activities of both UDP-3-O-(R-3-hydroxymyristoyl)-glucosamine N-acyltransferase and lipid A 4′ kinase (the sixth step of lipid A biosynthesis) were also shown to be decreased in strains with firA mutations (Roy et al., Mutations in firA, encoding the second acyltransferase in lipopolysaccharide biosynthesis, affect multiple steps in lipopolysaccharide biosynthesis. J. Bacteriol., 176:1639-46, 1994).

Once the strain of microorganism for use in the present invention has been attenuated by any of the methods known in the art, including any of those discussed above, it is desirable to maintain the stability of the attenuated microorganism. Maintaining the stability of the attenuated phenotype is important, because it essential that the strain does not revert to a more virulent phenotype during administration of the composition that comprises the attenuated microorganism. Such stability can be obtained, for example, with the use of non-reverting mutations on the chromosomal level (e.g., by deleting the whole virulence gene, or a significant portion thereof). Another means of ensuring the stability of the attenuated phenotype is to engineer the microorganism such that it is attenuated in more than one manner. For example, mutations may be introduced into the microorganism's genetic material, including a mutation in the pathway for lipid A production, such as the firA null mutation (Hirvas et al., Mutants carrying conditionally lethal mutations in outer membrane genes omsA and firA (ssc) are phenotypically similar, and omsA is allelic to firA.EMBO J., 10: 1017-23, 1991), plus one or more mutations to auxotrophy for one or more nutrients or metabolites, such as uracil biosynthesis, purine biosynthesis, and arginine biosynthesis (Bochner, et al., Positive selection for loss of tetracycline resistance. J. Bacteriol., 143:926-33, 1980). In one embodiment of the present invention, the microorganism is auxotrophic for uracil, aromatic amino acids, isoleucine, and valine, and synthesizes an altered lipid A.

In a further embodiment of the present invention, the engineered microorganism remains sensitive to as many antibiotics as possible. In a more preferred embodiment, the microorganism does not carry any antibiotic-resistance markers. This may create a problem in respect of the maintenance of stability of the engineered microorganism, because of a lack of selective pressure. However, there are a number of techniques by which the stability of the microorganism may be maintained without resorting to antibiotic resistance. For example, the stability of exogenous nucleic acids in the microorganism may be maintained through a balanced lethal system. In a balanced lethal system, the nucleic acid construct which carries the exogenous nucleic acids (usually episomal contructs, such as plasmids) encodes for a function that compensates for a deficiency in the microorganism (e.g., a metabolism defect), such that the presence of the construct is essential for the survival of the microorganism (Galan et al., Cloning and characterization of the asd gene of Salmonella typhimurium: use in stable maintenance of recombinant plasmids in Salmonella vaccine strains. Gene, 94:29-35, 1990).

In the composition of the present invention, the microorganism has, on its cell surface, at least one exogenous molecule. As used herein, the term “molecule” refers to the smallest particle of any substance that retains the chemical and physical properties of the substance, and is composed of two or more atoms, and includes a group of like or different atoms held together by chemical forces. As further used herein, an “exogenous” molecule is one that originates or arises outside the microorganism.

The molecule of the present invention may be any molecule that may be found on, expressed on, or attached onto, the surface of a microorganism. By way of example, the molecule may be any peptide (e.g., a polypeptide), saccharide (e.g., a polysaccharide), lipid (e.g., a glycolipid), a peptidoglycan, or any combination of peptides, saccharides, and lipids. Furthermore, the exogenous molecule may have any activity, function, or purpose. For example, the exogenous molecule may function as an antibody, an enzyme, a ligand, or a receptor. The exogenous molecule of the present invention is capable of binding to a receptor or other antigen on the surface of a target cell. In one embodiment of the present invention, the exogenous molecule is a polypeptide or a fragment thereof. In a preferred embodiment, the exogenous molecule is an antigen-binding polypeptide that specifically binds to an antigen on the surface of a target cell.

The term “polypeptide”, as used herein, includes proteins, polypeptides, peptides, and variants thereof. The variants preferably have greater than about 75% homology with the naturally-occurring polypeptide sequence, more preferably have greater than about 80% homology, even more preferably have greater than about 85% homology, and, most preferably, have greater than about 90% homology with the polypeptide sequence. In some embodiments, the homology may be as high as about 93-95%, 98%, or 99%. These variants may be substitutional, insertional, or deletional variants. The variants may also be chemically-modified derivatives: polypeptides which have been subjected to chemical modification, but which retain the biological characteristics of the naturally-occurring polypeptide.

Examples of exogenous polypeptides for use in the present invention include, but are not limited to, antibodies (e.g., IgA, IgD, IgE, IgG, IgM, and single-chain antibodies), fragments of antibodies (including F′ab fragments, such as scFv), ligands (e.g., cell-surface polypeptides, polysaccharide-polypeptides, peptidoglycans, lipid-polypeptides, and derivatives thereof), and receptors. In one embodiment of the present invention, the exogenous polypeptide is an antibody. In preferred embodiments of the present invention, the antibody is a mammalian antibody (e.g., a human antibody) or a chimeric antibody (e.g., a humanized antibody). More preferably, the antibody is a human or humanized antibody. As used herein, the term “humanized antibody” refers to a genetically-engineered antibody in which the minimum portion of an animal antibody (e.g., an antibody of a mouse, rat, pig, goat, or chicken) that is generally essential for its specific functions is “fused” onto a human antibody. In general, a humanized antibody is 1-25%, preferably 5-10%, animal; the remainder is human. Humanized antibodies usually initiate minimal or no response in the human immune system. Methods for expressing fully human or humanized antibodies in organisms other than human are well known in the art (see, e.g., U.S. Pat. No. 6,150,584, Human antibodies derived from immunized xenomice; U.S. Pat. No. 6,162,963, Generation of xenogenetic antibodies; and U.S. Pat. No. 6,479,284, Humanized antibody and uses thereof). In another embodiment of the present invention, the antibody is a single-chain antibody. In a preferred embodiment, the single-chain antibody is a human or humanized single-chain antibody. In another preferred embodiment of the present invention, the antibody is a murine antibody.

The exogenous molecule of the present invention may be produced by manipulation of the microorganism's cellular components, such that the microorganism itself ultimately produces the exogenous molecule. For example, the exogenous polypeptide (or a fragment thereof) of the present invention may be produced by genetically manipulating a microorganism's chromosomal DNA to contain DNA sequences encoding the polypeptide. In one embodiment of the invention, these DNA sequences are controlled by endogenous expression-regulation mechanisms. In another embodiment of the invention, these DNA sequences form parts of expression cassettes which contain exogenous expression-regulation elements (e.g., a promoter and terminator). The exogenous polypeptide (or fragment thereof) of the present invention also may be encoded by an expression vector, such as an episomal expression vector. The expression vector may contain or encode the agent of the present invention.

Where the exogenous molecule of the present invention is a polypeptide, the microorganism may be engineered to express the exogenous molecule transiently. Transient expression allows for greater control of the amount of exogenous polypeptide produced by the microorganism. Thus, expression can continue for a selected period of time, and can then be shut off. Accordingly, the composition of the present invention has an advantage over standard gene therapy techniques, in that expression of the exogenous polypeptide may be turned off before the composition causes damage in, or induces the development of a disease in, a subject into whom the composition has been introduced.

Expression of the exogenous polypeptide may be controlled by methods known in the art, including the use of attenuators, downregulators, inhibitors, and other molecules known to inhibit protein expression. By way of example, where the composition of the present invention is administered to a subject, such that the composition expresses an exogenous molecule in the subject, this expression may be shut off in vivo by subsequently administering to the subject an attenuator, downregulator, inhibitor, or other molecule that will inhibit expression of the exogenous molecule. Control of expression of the exogenous molecule is also advantageous, in that it allows for specific targeting to one type of target cell (e.g., tumor cells), thereby minimizing toxicity or harmful side-effects in a subject to whom the composition is administered.

Alternatively, the exogenous molecule of the present invention, may be synthesized, and then linked to the surface of the microorganism in vitro. For example, the exogenous polypeptide (or fragment thereof) may be synthesized, and then linked to the surface of the microorganism (covalently or non-covalently) in vitro. However, exogenous polypeptides (such as antibodies), when synthesized in microorganisms (such as E. coli, Salmonella, and Shigella), are not usually localized on the cell surface. Accordingly, it is necessary to design the expression constructs of the present invention such that the microorganism-produced exogenous polypeptides are displayed on the microorganism's cell surface. A number of displaying systems have been developed, and are readily available for the purposes of the present invention. For example, a Lpp-OmpA fusion vehicle system has been widely used to display antibodies (e.g., scFv) on the surface of bacteria (Earhart, C. F., Use of an Lpp-OmpA fusion vehicle for bacterial surface display. Methods Enzymol., 326:506-16, 2000; Daugherty et al., Development of an optimized expression system for the screening of antibody libraries displayed on the Escherichia coli surface. Protein Eng., 12:613-21, 1999; Georgiou et al., Display of β-lactamase on the Escherichia coli surface: outer membrane phenotypes conferred by Lpp′-OmpA′-β-lactamase fusions. Protein Eng., 9:239-47, 1996; Francisco et al., Production and fluorescence-activated cell sorting of Escherichia coli expressing a functional antibody fragment on the external surface. Proc. Natl. Acad. Sci. USA, 90:10444-448, 1993).

Feldhaus et al. have described a system for displaying human antibodies on the surface of yeast (Feldhaus et al., Flow-cytometric isolation of human antibodies from a nonimmune Saccharomyces cerevisiae surface display library. Nat. Biotechnol., 21(2):163-70, 2003). Other display systems include, without limitation, the Ag43, CotB, fimbrillin, flagellin, intimin, LamB, PhoE, and TraT systems. See, e.g., Kjaergaard et al., Antigen 43-mediated autotransporter display, a versatile bacterial cell surface presentation system. J. Bacteriol., 184:4197-204, 2002; Isticato et al., Surface display of recombinant proteins on Bacillus subtilis spores. J. Bacteriol., 183:6294-301, 2001; Chistmann et al., Epitope mapping and affinity purification of monospecific antibodies by Escherichia coli cell surface display of gene-derived random peptide libraries. J. Immunol. Methods, 257:163-73, 2001; Boder et al., Yeast surface display for directed evolution of protein expression, affinity, and stability. Methods Enzymol., 328:430-44, 2000; Stahl et al., Bacterial surface display: trends and progress. Trends Biotechnol., 15:185-92, 1997; Samuelson et al., Cell surface display of recombinant proteins on Staphylococcus carnosus. J. Bacteriol., 177:1470-6, 1995; Hofnung, Expression of foreign polypeptides at the Escherichia coli cell surface. Methods Cell Biol., 34:77-105, 1991; Harrison et al., Presentation of foreign antigenic determinants at the bacterial cell surface using the TraT lipoprotein. Res. Microbiol., 141:1009-12, 1990; Agterberg et al., Outer membrane PhoE protein of Escherichia coli as a carrier for foreign antigenic determinants: immunogenicity of epitopes of foot-and-mouth disease virus. Vaccine, 8:85-91, 1990; Newton et al. Immune response to cholera toxin epitope inserted in Salmonella flagellin. Science, 244:70-72, 1989; Hedegaard et al., Type 1 fimbriae of Escherichia coli as carriers of heterologous antigenic sequences. Gene, 85:115-24, 1989; and Charbit et al., Presentation of two epitopes of the preS2 region of hepatitis B virus on live recombinant bacteria. J. Immunol., 139:1658-64, 1987.

The composition of the present invention delivers an agent to a target cell. The target cell may be any cell of a mammal, including wild animals (e.g., primates, ungulates, rodents, felines, and canines), domestic animals (e.g., dog, cat, chicken, duck, goat, pig, cow, and sheep), and humans. In a preferred embodiment, the target cell is a human cell. The target cell may be in situ, within the mammal from which it is derived, or ex vivo.

In one embodiment of the present invention, the target cell is a cell of a neoplasm or neoplasia (i.e., is a “neoplastic cell”). As used herein, the term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17^{th} ed. (Whitehouse Station, N.J.: Merck Research Laboratories, 1999) 973-74, 976, 986, 988, 991). In a preferred embodiment of the present invention, the target cell is derived from a solid tumor (i.e., is a solid-tumor cell). More preferably, the target cell is derived from a colon tumor (i.e., is a colon-tumor cell).

In another embodiment of the present invention, the target cell expresses carcinoembryonic antigen (CEA). Examples of CEA-expressing cells include, without limitation, a bowel cancer cell, a breast cancer cell, a cervical cancer cell, a colon cancer cell, an esophageal cancer cell, a head cancer cell, a liver cancer cell, a lung cancer cell, a neck cancer cell, an ovarian cancer cell, a pancreatic cancer cell, and a stomach cancer cell. In a preferred embodiment of the present invention, the CEA-expressing cell is a colon cancer cell.

The target cell of the present invention has, on its surface, an antigen to which the exogenous molecule binds. As used herein, the term “antigen” refers to any molecule against which a host is capable of mounting an immune response, and includes, without limitation, polypeptide antigens (including glycoproteins and lipoproteins), polysaccharide antigens, lipid antigens, any ligand or receptor on the surface of the target cell, and any co-stimulatory molecule (such as a cytokine receptor or a chemokine receptor) that activates an immune response.

As discussed above, the target cell of the present invention may be derived from a neoplasm (i.e., it is a neoplastic cell). Accordingly, in one embodiment of the invention, the antigen on the surface of the target cell is a neoplasm-specific antigen. The neoplasm-specific antigen may be any molecule against which a host is capable of mounting an immune response, including, without limitation, a polypeptide (e.g., a glycoprotein or lipoprotein), a polysaccharide, and a lipid. In a preferred embodiment of the present invention, the neoplasm-specific antigen is a solid-tumor-specific antigen. In another preferred embodiment of the present invention, the neoplasm-specific antigen is CAK1, CDK4, CDR2, carcinoembryonic antigen (CEA), disialoganglioside GD2, HER-2, large external antigen (LEA), MAGEs, MUC1, p21, podocalyxin, Ras, UK114, or WT1. Preferably, the antigen is CEA.

The composition of the present invention comprises a microorganism expressing an exogenous molecule and an agent. As used herein, the term “agent” shall include any protein, polypeptide, peptide, nucleic acid (including DNA, RNA, and genes), antibody, Fab fragment, F(ab!)₂ fragment, molecule, compound, antibiotic, drug, and any combinations thereof. A Fab fragment is a univalent antigen-binding fragment of an antibody, which is produced by papain digestion. A F(ab!)₂ fragment is a divalent antigen-binding fragment of an antibody, which is produced by pepsin digestion. The agent of the present invention may have any activity, function, or purpose. By way of example, the agent may be a diagnostic agent, a labelling agent, a preventive agent, or a therapeutic or pharmacologic agent.

As used herein, a “diagnostic agent” is an agent that is used to detect a disease, disorder, or illness, or is used to determine the cause thereof. As further used herein, a “labelling agent” is an agent that is linked to, or incorporated into, a cell or molecule, to facilitate or enable the detection or observation of that cell or molecule. By way of example, the labelling agent of the present invention may be an imaging agent or detectable marker, and may include any of those chemiluminescent and radioactive labels known in the art. The labelling agent of the present invention may be, for example, a nonradioactive or fluorescent marker, such as biotin, fluorescein (FITC), acridine, cholesterol, or carboxy-X-rhodamine (ROX), which can be detected using fluorescence and other imaging techniques readily known in the art. Alternatively, the labelling agent may be a radioactive marker, including, for example, a radioisotope, such as a low-radiation isotope. The radioisotope may be any isotope that emits detectable radiation, and may include ³⁵S, ³²p, ³H, radioiodide (125I- or ¹³¹I-), or ⁹⁹mTc-pertechnetate (⁹⁹ mTcO₄⁻). Radioactivity emitted by a radioisotope can be detected by techniques well known in the art. For example, gamma emission from the radioisotope may be detected using gamma imaging techniques, particularly scintigraphic imaging.

Additionally, as used herein, the term “preventive agent” refers to an agent, such as a prophylactic, that helps to prevent a disease, disorder, or illness in a subject. As further used herein, the term “therapeutic” refers to an agent that is useful in treating a disease, disorder, or illness (e.g., a neoplasm) in a subject.

In one embodiment of the present invention, the agent is a nucleic acid, a polypeptide, a polysaccharide, a lipid, a pro-drug, or an anti-tumor compound. In a preferred embodiment of the present invention, the agent is a therapeutic nucleic acid. As used herein, a “nucleic acid” or “polynucleotide” includes a nucleic acid, an oligonucleotide, a nucleotide, a polynucleotide, or any fragment thereof. The nucleic acid or polynucleotide may be double-stranded or single-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, and xanthine hypoxanthine. The nucleic acid or polynucleotide may be combined with a carbohydrate, a lipid, a protein, or other materials.

The “complement” of a nucleic acid refers, herein, to a nucleic acid molecule which is completely complementary to another nucleic acid, or which will hybridize to the other nucleic acid under conditions of high stringency. High-stringency conditions are known in the art. See, e.g., Maniatis et al., Molecular Cloning: A Laboratory Manual, 2^{nd} ed. (Cold Spring Harbor: Cold Spring Harbor Laboratory, 1989) and Ausubel et al., eds., Current Protocols in Molecular Biology (New York, N.Y.: John Wiley & Sons, Inc., 2001). Stringent conditions are sequence-dependent, and may vary depending upon the circumstances. Additionally, as used herein, the term “cDNA” refers to an isolated DNA polynucleotide or nucleic acid molecule, or any fragment, derivative, or complement thereof. It may be double-stranded or single-stranded, it may have originated recombinantly or synthetically, and it may represent coding and/or noncoding 5′ and 3′ sequences.

The nucleic acid agent of the present invention may be a plasmid, although it is to be understood that other types of nucleic acid agents, such as cosmids and phagemids, may also be used for the purposes of the present invention (particularly as therapeutic agents). The term “plasmid”, as used herein, refers generally to circular double-stranded DNA which is not bound to a chromosome. The DNA may be a chromosomal or episomal-derived plasmid. The plasmid of the present invention may optionally contain a terminator of transcription; a promoter; and/or a discrete series of restriction-endonuclease recognition sites, located between the promoter and the terminator. In the plasmid, a polynucleotide insert of interest (e.g., one encoding a therapeutically-active agent) should be operatively linked to an appropriate promoter, such as its native promoter or a host-derived promoter, such as the E. coli lacZ promoters, the trp and tac promoters, the T3 and T7 promoters, or the CMV promoters. Other suitable promoters will be known to the skilled artisan.

In one embodiment of the present invention, the nucleic acid (e.g., plasmid) encodes or comprises at least one gene-silencing cassette. It is well understood in the art that a gene may be silenced at a number of stages, including, without limitation, pre-transcription silencing, transcription silencing, translation silencing, post-transcription silencing, and post-translation silencing. In a preferred embodiment, the gene-silencing cassette is a gene-knockout cassette. Methods to construct gene-knockout cassettes are well known in the art. See, e.g., U.S. Pat. No. 6,503,712, Methods and compositions for preparing a genomic library for knockout targeting vectors; U.S. Pat. No. 6,461,864, Methods and vector constructs for making non-human animals which ubiquitously express a heterologous gene; Steinbrecher et al., Targeted inactivation of the mouse guanylin gene results in altered dynamics of colonic epithelial proliferation. Am. J. Pathol., 161:2169-78, 2002; Arakawa et al., Mutant loxP vectors for selectable marker recycle and conditional knock-outs. BMC Biotechnol., 1:7, 2001; Frengen et al., Modular bacterial artificial chromosome vectors for transfer of large inserts into mammalian cells. Genomics, 68:118-26, 2000; and Westphal et al., Transposon-generated ‘knock-out’ and ‘knock-in’ gene-targeting constructs for use in mice. Curr. Biol., 7:530-3, 1997.

By way of example, an essential neoplastic gene in the target cell (e.g., an oncogene) may be knocked out by the cassette through homologous recombination. Homologous recombination is a process that relies on the tendency of nucleic acids to base pair with substantially-complementary sequences. This base pairing functions to facilitate the interaction of two separate nucleic acids, bringing two complementary sequences into close proximity. Once the two nucleic acids are in close proximity, the host cell provides a mechanism to catalyze the strand breakage and repair which results when one complementary sequence is replaced by the other. The term “substantially complementary”, as used herein, refers to a sequence that is complementary to a sequence that substantially corresponds to a reference sequence. It is understood in the art that, in general, targeting efficiency increases with the length of the targeting transgene portion (i.e., homology region) that is substantially complementary to a reference sequence present in the target nucleic acid.

In another embodiment of the present invention, the gene-silencing cassette encodes a post-transcription gene-silencing composition, such as antisense RNA or RNAi. Antisense RNA is an RNA molecule with a sequence complementary to a specific RNA transcript, or mRNA, whose binding prevents further processing of the transcript or translation of the mRNA.

Antisense molecules may be generated, synthetically or recombinantly, with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are sensitive to nucleases, and have half-lives of 15-30 min in serum. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein et al., Oligodeoxynucleotides as inhibitors of gene expression: a review. Cancer Res., 48:2659-68, 1998). Antisense molecules designed to bind to the entire mRNA may be made by inserting cDNA into an expression plasmid in the opposite or antisense orientation. Antisense molecules may also function by preventing translation initiation factors from binding near the 5′ cap site of the mRNA, or by interfering with interaction of the MRNA and ribosomes. See, e.g., U.S. Pat. No. 6,448,080, Antisense modulation of WRN expression; U.S. patent application No. 2003/0018993, Methods of gene silencing using inverted repeat sequences; U.S. Patent application No., 2003/0017549, Methods and compositions for expressing polynucleotides specifically in smooth muscle cells in vivo; Tavian et al., Stable expression of antisense urokinase mRNA inhibits the proliferation and invasion of human hepatocellular carcinoma cells. Cancer Gene Ther., 10:112-20, 2003; Maxwell and Rivera, Proline oxidase induces apoptosis in tumor cells and its expression is absent or reduced in renal carcinoma. J. Biol. Chem., e-publication ahead of print, 2003; Ghosh et al., Role of superoxide dismutase in survival of Leishmania within the macrophage. Biochem. J., 369:447-52, 2003; and Zhang et al., An anti-sense construct of full-length ATM cDNA imposes a radiosensitive phenotype on normal cells. Oncogene, 17:811-8, 1998.

RNA interference (RNAi) is an RNA-mediated, sequence-specific gene-silencing mechanism. RNAi, a double-stranded (ds) interference RNA, was discovered by Guo and Kemphues in 1995, when they reported that both the sense and antisense strands of test oligonuleotides disrupted the expression of par-1 in Caenorhabditis elegans, following injection into a cell (Guo et al., Par-1, A gene required for establishing polarity in C. elegans embryos, encodes a putative Ser/Thr kinase that is asymmetrically distributed. Cell, 81:611-20, 1995). In 1998, Fire et al. clearly proved the existence and efficacy of RNAi by injecting into the gut of C. elegans a dsRNA that had been prepared in vitro (Fire et al., Potent and specific genetic interference by double-stranded RNA in Caenorhabditis elegans, Nature, 391:806-11, 1998). The injection of dsRNA into C. elegans resulted in loss of expression of the homologous target gene, not only throughout the worm, but also in its progeny. It is now well accepted that the phenomenon of RNAi is ubiquitous among bacteria, fungi, plants, and animals.

As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment, or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

In one embodiment of the present invention, RNAi is produced in vivo by an expression vector containing a gene-silencing cassette coding for RNAi. See, e.g., U.S. Pat. No. 6,278,039, C. elegans deletion mutants; U.S. patent application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes. In another embodiment of the present invention, RNAi is produced in vitro, synthetically or recombinantly, and transferred into the microorganism using standard molecular-biology techniques. Methods of making and transferring RNAi are well known in the art. See, e.g., Ashrafi et al., Genome-wide RNAi analysis of Caenorhabditis elegans fat regulatory genes. Nature, 421:268-72, 2003; Cottrell et al., Silence of the strands: RNA interference in eukaryotic pathogens. Trends Microbiol., 11:37-43, 2003; Nikolaev et al., Parc. A Cytoplasmic Anchor for p53. Cell, 112:29-40, 2003; Wilda et al., Killing of leukemic cells with a BCR/ABL fusion gene RNA interference (RNAi). Oncogene, 21:5716-24, 2002; Escobar et al., RNAi-mediated oncogene silencing confers resistance to crown gall tumorigenesis. Proc. Natl. Acad. Sci. USA, 98:13437-42, 2001; and Billy et al., Specific interference with gene expression induced by long, double-stranded RNA in mouse embryonal teratocarcinoma cell lines. Proc. Natl. Acad. Sci. USA, 98:14428-33, 2001. This approach may have an application in the repair of developmental deficits in stem cells and/or pluripotent cells.

In another embodiment of the present invention, the plasmid is an expression plasmid. The expression plasmid may contain sites for transcription initiation, termination, and, optionally, in the transcribed region, a ribosome-binding site for translation. The coding portions of the mature transcripts expressed by the plasmid may include a translation-initiating codon at the beginning, and a termination codon appropriately positioned at the end of the polypeptide to be translated.

In one embodiment of the present invention, the genes to be expressed from the expression plasmids are under the specific regulatory control of certain types of promoters. In one embodiment, these promoters are constitutive promoters. Genes under the control of these constitutive promoters will be expressed continually. In another embodiment, the promoters are inducible promoters. Genes under the control of these inducible promoters will be expressed only upon the presence of an inducer molecule or the absence of an inhibitor molecule. In yet another embodiment, the promoters are cell-type-specific promoters. Genes under the control of cell-type-specific promoters will be expressed only in certain cell types. In still another embodiment, the promoters are tumor developmental stage-specific promoters. Genes under the control of tumor developmental stage-specific promoters will be expressed only in neoplastic cells in certain developmental stages.

In another embodiment of the present invention, gene expression is controlled by a microorganism promoter which is activated in, or upon contact with, specific target cells. In a preferred mode of this embodiment, a bacterial promoter is activated primarily in, or upon contact with, specific target cells. In another embodiment of the present invention, bacterial gene expression is controlled by a promoter which is activated only in specific neoplastic cells, particularly solid-tumor cells.

The agent of the present invention also may be a polypeptide. By way of example, the agent may be a therapeutic pro-apoptotic factor, anti-proliferation factor, immuno-enhancing factor, pro-drug converting enzyme, or antibody, or any fragment thereof. In one embodiment of the present invention, the polypeptide is modified by glycosylation or lipid linkage.

The term “pro-apoptotic factor”, as used herein, refers to a factor which causes apoptosis and/or necrosis of the target cell (e.g., a neoplastic cell, such as a solid-tumor cell). A large number of pro-apoptotic factors have been isolated in the last few decades. Examples of pro-apoptotic factors, or apoptosis-/necrosis-inducing factors, include, without limitation, AIF, Apaf-1, Apo2L/TRAIL, Bax, Bik, caspases, cytochrome C, fas/CD95, metallothionein-III, Perforin, and tumor suppressors (such as p53, RB, and p27). See, e.g., Cheng et al., Activated protein C blocks p53-mediated apoptosis in ischemic human brain endothelium and is neuroprotective. Nat. Med., e-publication ahead of print, 2003; Carneiro et al., p27 deficiency desensitizes Rb-/-cells to signals that trigger apoptosis during pituitary tumor development. Oncogene, 22:361-69, 2003; Heinrichs et al., Apoptosis or growth arrest: modulation of the cellular response to p53 by proliferative signals. Oncogene, 22:555-71, 2003; Lu et al., Activation of multiple caspases and modification of cell surface fas (CD95) in proteasome inhibitor-induced apoptosis of rat natural killer cells. J. Cell. Biochem., 88:482-492, 2003; Shi, Y., Mechanisms of caspase activation and inhibition during apoptosis. Mol. Cell, 9:459-70, 2002; Morishima et al., An endoplasmic reticulum stress-specific caspase cascade in apoptosis. Cytochrome c-independent activation of caspase-9 by caspase-12. J. Biol. Chem., 277:34287-94; 2002; Yu et al., Mediation of poly(ADP-ribose) polymerase-1-dependent cell death by apoptosis-inducing factor. Science, 297:259-63, 2002; Zou et al., Systemic tumor suppression by the proapoptotic gene bik. Cancer Res., 62:8-12, 2002; Zhou et al., Radiation and the Apo2L/TRAIL apoptotic pathway preferentially inhibit the colonization of premalignant human breast cells overexpressing cyclin D1. Cancer Res., 60(10):2611-5, 2000; Hymowitz et al., Triggering cell death: the crystal structure of Apo2L/TRAIL in a complex with death receptor 5. Mol. Cell, 4:563-71, 1999; Hsu et al., Induction of p21 (CIP1/Waf1) and activation of p34 (cdc2) involved in retinoic acid-induced apoptosis in human hepatoma Hep3 B cells. Exp. Cell Res., 248:87-96, 1999; Quaife et al., Ectopic expression of metallothionein-III causes pancreatic acinar cell necrosis in transgenic mice. Toxicol. Appl. Pharmacol., 148:148-57, 1998; Laochumroonvorapong et al., Perforin, a cytotoxic molecule which mediates cell necrosis, is not required for the early control of mycobacterial infection in mice. Infect. Immun., 65:127-32, 1997; Wen et al., Cleavage of focal adhesion kinase by caspases during apoptosis. J. Biol. Chem., 272:26056-61, 1997; Zou et al., Apaf-1, a human protein homologous to C. elegans CED-4, participates in cytochrome c-dependent activation of caspase-3. Cell, 90:405-13, 1997; Johnstone et al., A novel repressor, par-4, modulates transcription and growth suppression functions of the Wilms' tumor suppressor WT1. Mol. Cell Biol., 16:6945-56, 1996; Liu et al., Induction of apoptotic program in cell-free extracts: requirement for dATP and cytochrome c. Cell, 86:147-57, 1996; and Oltvai et al., Bcl-2 heterodimerizes in vivo with a conserved homolog, Bax, that accelerates programmed cell death. Cell, 74:609-19, 1993.

An “anti-proliferation factor”, for the purposes of the present invention, is a factor that inhibits the growth and proliferation of the target cell (e.g., a neoplastic cell, such as a solid-tumor cell). Examples of anti-proliferation factors include, without limitation, human papillomavirus E6 and E7 proteins, heregulin, and adhesion molecules (such as cadherin and C-CAM). See, e.g., DeFilippis et al., Endogenous human papillomavirus E6 and E7 proteins differentially regulate proliferation, senescence, and apoptosis in HeLa cervical carcinoma cells. J. Virol., 77:1551-63, 2003; Puricelli et al., Heregulin inhibits proliferation via ERKs and phosphatidyl-inositol 3-kinase activation but regulates urokinase plasminogen activator independently of these pathways in metastatic mammary tumor cells. Int. J. Cancer, 100:642-53, 2002; Shah et al., The role of cadherin, β-catenin, and AP-1 in retinoid-regulated carcinoma cell differentiation and proliferation. J. Biol. Chem., 277:25313-22, 2002; Singer et al., The tumor growth-inhibiting cell adhesion molecule CEACAM1 (C-CAM) is differently expressed in proliferating and quiescent epithelial cells and regulates cell proliferation. Cancer Res., 60:1236-44, 2000; and Bouterfa et al., Retinoids inhibit human glioma cell proliferation and migration in primary cell cultures but not in established cell lines. Neurosurgery, 46:419-30, 2000.

As used herein, the term “immuno-enhancing factor” refers to a factor that enhances the responses of a host's immune system to the target cell (e.g., a neoplastic cell, such as a solid-tumor cell). Examples of inumuno-enhancing factors for use in the present invention include, without limitation, cytokines (such as tumor necrosis factors-α, β; interferons-α,β, and γ; interleukins; CD30L; CD40L; OX40L; 4-1BBL; and LICOS) and chemokines (such as eotaxin, fractalkine, IP-10, lymphotactin, Mig, MIP-1α, MIP-1β, RANTES, and TARC). See, e.g., Khayyamian et al., ICOS-ligand, expressed on human endothelial cells, costimulates Th1 and Th2 cytokine secretion by memory CD4+ T cells. Proc. Natl. Acad. Sci. USA, 99:6198-203, 2002; Clodi et al., Expression of CD40 ligand (CD154) in B and T lymphocytes of Hodgkin disease: potential therapeutic significance. Cancer, 94:1-5, 2002; Woodward et al., Stimulation and inhibition of uveal melanoma invasion by HGF, GRO, IL-1α and TGF-β. Invest Ophthalmol. Vis. Sci., 43:3144-52, 2002; van Deventer et al., Transfection of macrophage inflammatory protein 1 α into B16 F10 melanoma cells inhibits growth of pulmonary metastases but not subcutaneous tumors. J. Immunol., 169:1634-39, 2002; Cairns et al., Lymphotactin expression by engineered myeloma cells drives tumor regression: mediation by CD4+ and CD8+ T cells and neutrophils expressing XCR1 receptor. J. Immunol., 167:57-65, 2001; Venters et al., Tumor necrosis factor-α induces neuronal death by silencing survival signals generated by the type I insulin-like growth factor receptor. Ann. N. Y. Acad. Sci., 917:210-20, 2000; van den Berg et al., High expression of the CC chemokine TARC in Reed-Sternberg cells. A possible explanation for the characteristic T-cell infiltratein Hodgkin's lymphoma. Am. J. Pathol., 154:1685-91, 1999; Tannenbaum et al., The CXC chemokines IP-10 and Mig are necessary for IL-12-mediated regression of the mouse RENCA tumor. J. Immunol., 161:927-32, 1998; and Rothenberg et al., Murine eotaxin: an eosinophil chemoattractant inducible in endothelial cells and in interleukin 4-induced tumor suppression. Proc. Natl. Acad. Sci. USA, 92:8960-4, 1995.

A “pro-drug converting enzyme”, as referred to herein, is a polypeptide that converts a pro-drug to an active functional drug to treat the target cell (e.g., a neoplastic cell, such as a solid-tumor cell). The term “pro-drug”, as used herein, refers to any compound that may have less biological activity than a drug, but, when administered to a subject, generates the effective drug substance, either as a result of a spontaneous chemical reaction or by enzyme catalysis or metabolic reaction. Pro-drug converting enzymes are currently widely employed in gene therapy for use in the treatment of malignant cancers. See, e.g., Mullen et al., Tumors expressing the cytosine deaminase suicide gene can be eliminated in vivo with 5-fluorocytosine and induce protective immunity to wild type tumor. Cancer Res., 54:1503-06, 1994; Austin et al., A first step in the development of gene therapy for colorectal carcinoma: cloning, sequencing, and expression of Escherichia coli cytosine deaminase. Mol. Pharmacol., 43:380-87, 1993; Vile et al., Use of tissue-specific expression of the herpes simplex virus thymidine kinase gene to inhibit growth of established murine melanomas following direct intratumoral injection of DNA. Cancer Res., 53:3860-64, 1993; Moolten et al., Curability of tumors bearing herpes thymidine kinase genes transferred by retroviral vectors. J. Natl. Cancer Inst., 82(4):297-300, 1990; Wagner et al., Nucleotide sequence of the thymidine kinase gene of herpes simplex virus type 1. Proc. Natl. Acad. Sci. USA, 78:1441-45, 1981.

Pro-drug converting enzymes have been expressed in a number of microorganisms. For example, Herpes simplex virus thymidine kinase (TK) phosphorylates the non-toxic pro-drugs, acyclovir and ganciclovir, rendering them toxic via their incorporation into the genomic DNA of neoplastic cells (Kokoris et al., Characterization of Herpes Simplex Virus type 1 thymidine kinase mutants engineered for improved ganciclovir or acyclovir activity. Protein Sci., 11:2267-72, 2002; Black et al., Creation of drug-specific herpes simplex virus type 1 thymidine kinase mutants for gene therapy. Proc. Natl. Acad. Sci. USA, 93:3525-29, 1996). Another example of a pro-drug converting enzyme is bacterial cytosine deaminase (CD), which converts the non-toxic pro-drug, 5-fluorocytosine (5-FC), into 5-fluorouracil (5-FU), a well-known anti-cancer drug (Mullen et al., Transfer of the bacterial gene for cytosine deaminase to mammalian cells confers lethal sensitivity to 5-fluorocytosine: a negative selection system. Proc. Natl. Acad. Sci. USA, 89:33-37, 1992; Nishiyama et al., Antineoplastic effects in rats of 5-fluorocytosine in combination with cytosine deaminase capsules. Cancer Res., 45:1753-61, 1985).

Additionally, the pro-drug converting enzyme, cytochrome p450 oxidoreductase, has been expressed in Salmonella typhimurium, and has thereby conferred sensitivity to mitomycin (Simula et al., Heterologous expression of drug-metabolizing enzymes in cellular and whole animal models. Toxicology, 82:3-20, 1993). Further examples of pro-drug converting enzymes include, without limitation, carboxypeptidase A, carboxypeptidase G2, β-glucuronidase, β-lactamase, nitroreductase, penicillin-V-amidase, and penicillin-G-amidase. See, e.g., Vrudhula et al., Prodrugs of doxorubicin and melphalan and their activation by a monoclonal antibody-penicillin-G amidase conjugate. J. Med. Chem., 36:919-23, 1993; Meyer et al., Site-specific prodrug activation by antibody-β-lactamase conjugates: regression and long-term growth inhibition of human colon carcinoma xenograft models. Cancer Res., 53:3956-63, 1993; Haenseler et al., Activation of methotrexate-alpha-alanine by carboxypeptidase A-monoclonal antibody conjugate. Biochemistry, 31:891-97, 1992; Bignami et al., N-(4′-hydroxyphenylacetyl)palytoxin: a palytoxin prodrug that can be activated by a monoclonal antibody-penicillin G amidase conjugate. Cancer Res., 52:5759-64, 1992; Haisma et al., Analysis of a conjugate between anti-carcinoembryonic antigen monoclonal antibody and alkaline phosphatase for specific activation of the prodrug etoposide phosphate. Cancer Immunol. Immunother., 34:343-48, 1992; Haisma et al., A monoclonal antibody-β-glucuronidase conjugate as activator of the prodrug epirubicin-glucuronide for specific treatment of cancer. Br. J. Cancer, 66:474-78, 1992; Roffler et al., Anti-neoplastic glucuronide prodrug treatment of human tumor cells targeted with a monoclonal antibody-enzyme conjugate. Biochem. Pharmacol., 42:2062-65, 1991; Kerr et al., Antibody-penicillin-V-amidase conjugates kill antigen-positive tumor cells when combined with doxorubicin phenoxyacetamide. Cancer Immunol. Immunother., 31:202-06, 1990; Springer et al., Novel prodrugs which are activated to cytotoxic alkylating agents by carboxypeptidase G2. J. Med. Chem., 33:677-81, 1990; Knox et al., A new cytotoxic, DNA interstrand crosslinking agent, 5-(aziridin-1-yl)-4-hydroxylamino-2-nitrobenzamide, is formed from 5-(aziridin-1-yl)-2,4-dinitrobenzamide (CB 1954) by a nitroreductase enzyme in Walker carcinoma cells. Biochem. Pharmacol., 37:4661-69, 1988; and Bagshawe et al., A cytotoxic agent can be generated selectively at cancer sites. Br. J. Cancer, 58:700-03, 1988.

Where the agent of the present invention is a therapeutic pro-drug converting enzyme, the agent may further comprise the corresponding pro-drug itself. In this case, the composition of the present invention would be pre-loaded with the pro-drug, and the therapeutic agent of the composition would be a combination therapeutic agent comprising the enzyme and the pro-drug which it converts. Alternatively, where the therapeutic agent is a pro-drug converting enzyme, the therapeutic agent may comprise only the enzyme itself, and not the associated pro-drug. In this case, for the composition of the present invention to have any efficacy in a subject, the pro-drug that corresponds with the enzyme of the therapeutic agent would have to be administered to the subject in combination with the composition comprising the enzyme.

In yet another embodiment of the present invention, the therapeutic agent is an antibody. The skilled artisan will readily appreciate that antibodies that inhibit cell growth or proliferation, and/or interfere with normal cell metabolism, may be used to treat neoplastic cells (e.g., solid-tumor cells). Examples of such antibodies include, but are not limited to, antibodies against oncoproteins (such as pX, p21, and C-erbB-2), anti-apoptosis factors (such as bcl-2), growth factors and receptors thereof (such as EGF and EGFR), angiogenesis factors and receptors thereof (such as IL-17), signal transduction enzymes (such as PI3K, MEK, PKA, and PKC), cell-cycle regulators (such as cyclins and cyclin-dependent kinases), enzymes required for DNA replication, repair, and RNA production (such as DNA and RNA polymerases), anti-oxidative enzymes (such as SOD), ion channels (such as Ca²⁺ and Na⁺ channels), glycolytic-pathway enzymes (such as hexokinase and pyruvate kinase), amino acid metabolism enzymes (such as aminotransferases), and nucleotide metabolism enzymes (such as thymidylate synthase, dihydrofolate reductase, IMP dehydrogenase, ribonucleotide reductase, and DNA methyltransferase). See, e.g., Nakamoto et al., Prevention of hepatocellular carcinoma development associated with chronic hepatitis by anti-fas ligand antibody therapy. J. Exp. Med., 196:1105-11, 2002; Rundle et al., Association between the ras p21 oncoprotein in blood samples and breast cancer. Cancer Lett., 185:71-78, 2002; Numasaki et al., Interleukin-17 promotes angiogenesis and tumor growth. Blood, e-publication ahead of print, 2002; Pedersen et al., Mitochondrial bound type II hexokinase: a key player in the growth and survival of many cancers and an ideal prospect for therapeutic intervention. Biochim. Biophys. Acta, 1555:14-20, 2002; Lee et al., The hepatitis B virus encoded oncoprotein pX amplifies TGF-β family signaling through direct interaction with Smad4: potential mechanism of hepatitis B virus-induced liver fibrosis. Genes Dev., 15:455-66, 2001; Sutterlin et al., The correlation of c-erbB-2 oncoprotein and established prognostic factors in human breast cancer. Anticancer Res., 20:5083-8, 2000; Hatse et al., Role of antimetabolites of purine and pyrimidine nucleotide metabolism in tumor cell differentiation. Biochem. Pharmacol., 58:539-55, 1999; and Cascales et al., Effects of an antitumoural rhodium complex on thioacetamide-induced liver tumor in rats. Changes in the activities of ornithine decarboxylase, tyrosine aminotransferase and of enzymes involved in fatty acid and glycerolipid synthesis. Biochem. Pharmacol., 35:2655-61, 1986.

The present invention further provides a vaccine comprising: (a) at least one microorganism that has, on its cell surface, at least one exogenous molecule that binds to an antigen on the surface of a target cell; (b) an agent; and (c) a pharmaceutically-acceptable carrier. The pharmaceutically-acceptable carrier must be “acceptable” in the sense of being compatible with the other ingredients of the composition, and not deleterious to the recipient thereof. The pharmaceutically-acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The vaccine of the present invention may be prepared by methods well-known in the pharmaceutical arts. For example, the vaccine may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the vaccine. Formulations of the vaccine may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered orally, topically, or by injection, including, but not limited to, intravenous, intraperitoneal, subcutaneous, intramuscular, and intratumoral (i.e. direct injection into the tumor) injection.

The vaccine of the present invention may be useful for administering an agent to a subject, including administration of a therapeutic agent to a subject to treat a variety of disorders, including cancer. The therapeutic agent is provided in an amount that is effective to treat the disorder in a subject to whom the vaccine is administered. This amount may be readily determined by the skilled artisan.

The present invention also provides a method for treating neoplasia in a subject in need of treatment, comprising administering to the subject a therapeutic composition in an amount effective to treat the neoplasia. As used herein, the “subject” is a mammal, including, without limitation, a cow, dog, human, monkey, mouse, pig, or rat. Preferably, the subject is a human. The neoplasia may be any of those described above, but is preferably a CEA-expressing tumor (e.g., a colon tumor).

The therapeutic composition for use in the method of the present invention comprises: (a) a microorganism that has, on its cell surface, at least one exogenous molecule that binds to an antigen on the surface of a neoplastic cell in the subject; and (b) a therapeutic agent. Optionally, the therapeutic composition also may comprise a pharmaceutically-acceptable carrier. The therapeutic composition may be any composition or vaccine of the present invention, as described above.

In the method of the present invention, the therapeutic composition is administered to a subject who has neoplasia in an amount effective to treat the neoplasia in the subject. As used herein, the phrase “effective to treat the neoplasia” means effective to ameliorate or minimize the clinical impairment or symptoms resulting from the neoplasia. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasia; or by limiting, suspending, terminating, or otherwise controlling the proliferation of cells in the neoplasm.

The amount of therapeutic composition that is effective to treat neoplasia in a subject will vary depending on the particular factors of each case, including the type of neoplasia, the stage of neoplasia, the subject's weight, the severity of the subject's condition, and the method of administration. These amounts can be readily determined by the skilled artisan. In general, the dosage of microorganism (within the therapeutic composition) to be administered to a subject may range from about 1 to 1×10⁹ c.f.u./kg, preferably from about 1×10² to 1×10⁷ c.f.u./kg, and, more preferably, from about 2×10² to 1×10⁶ c.f.u./kg.

In the method of the present invention, the therapeutic composition may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous, or subcutaneous administration), transdermal administration, and administration by osmotic pump. One preferred method of administration is parenteral administration, by intravenous or subcutaneous injection.

For oral administration, the formulation of the therapeutic composition may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the therapeutic composition may be combined with a sterile aqueous solution, which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation also may be delivered by any mode of injection, including any of those described above. Where a neoplasm is localized to a particular portion of the body of the subject, it may be desirable to introduce the therapeutic composition directly to that area by injection or by some other means (e.g., by introducing the therapeutic composition into the blood or another body fluid).

For transdermal administration, the therapeutic composition may be combined with skin penetration enhancers, such as propylene glycol, polyethylene glycol, isopropanol, ethanol, oleic acid, N-methylpyrrblidone, and the like, which increase the permeability of the skin to the therapeutic composition, and permit the therapeutic composition to penetrate through the skin and into the bloodstream. The therapeutic composition also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The therapeutic composition may be administered transdermally, at or near the site on the subject where the neoplasm is localized. Alternatively, the therapeutic composition may be administered transdermally at a site other than the affected area, in order to achieve systemic administration.

The therapeutic composition of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the therapeutic composition.

In accordance with the method of the present invention, the therapeutic composition may be administered to a subject who has neoplasia, either alone or in combination with one or more antineoplastic drugs used to treat neoplasias. Examples of antineoplastic drugs with which the therapeutic composition may be combined include, without limitation, carboplatin, cyclophosphamide, doxorubicin, etoposide, and vincristine. Additionally, when administered to a subject, the therapeutic composition may be combined with other neoplastic therapies, including, without limitation, surgical therapies, radiotherapies, gene therapies, and immunotherapies.

In one embodiment of the present invention, the therapeutic composition comprises a therapeutic agent that is localized in the microorganism of the therapeutic composition. For instance, where the therapeutic agent comprises a pro-drug converting enzyme, the therapeutic composition may be pre-loaded with the enzyme's associated pro-drug, or may lack the corresponding pro-drug. Where the therapeutic composition is not pre-loaded with the enzyme's associated pro-drug, the pro-drug would have to be co-administered to a subject along with the therapeutic composition comprising the pro-drug converting enzyme.

By way of example, the therapeutic agent in the therapeutic composition of the present invention may comprise the pro-drug converting enzyme, TK. Upon administration of ganciclovir (the enzyme's associated pro-drug) to a subject in need of treatment for neoplasia, the TK enzyme, which is localized in the microorganism of the therapeutic composition, phosphorylates ganciclovir in the periplasm of the microorganism. The phosphorylated ganciclovir, a toxic compound, passes out of the periplasm of the microorganism and into the cytoplasm and nucleus of the subject's neoplastic cell, where it is incorporated into the subject's cell DNA, thereby causing the death of the subject's neoplastic cell.

In another embodiment of the present invention, the therapeutic agents (e.g., polypeptides, including antibodies and pro-drug converting enzymes) are secreted to or into the subject's neoplastic cells. By way of example, techniques for constructing expression cassettes (chromosomal or episomal) to direct gene products to or into target cells, from a microorganism, are well known in the art. A general strategy involves fusing the polypeptide to be secreted to a signal sequence which is capable of directing the transport process. A large number of such signal sequences have been isolated.

In a preferred embodiment of the present invention, such sequences are N-terminal signal sequences containing hydrophobic transmembrane domains. These sequences serve to guide the protein through the membrane, and, optionally, are removed as, or after, the protein crosses the membrane. Prokaryotic and eukaryotic N-terminal signal sequences are similar, and it has been shown that eukaryotic N-terminal signal sequences are capable of functioning as secretion sequences in bacteria. An example of such an N-terminal signal sequence is the bacterial β-lactamase signal sequence, which is a well-studied sequence, and has been widely used to facilitate the secretion of polypeptides into the external environment.

In another preferred embodiment of the present invention, the signal sequences are C-terminal signal sequences, such as the hemolysin A (hlyA) signal sequences of E. coli. It has been established that the secretion signal is located in the last 60 amino acids of hlyA, and that transfer of this domain to other proteins can result in their direct secretion into surrounding media (Hui et al., A combinatorial approach toward analyzing functional elements of the Escherichia coli hemolysin signal sequence. Biochemistry, 41:5333-39, 2002; Koronakis et al., Isolation and analysis of the C-terminal signal directing export of Escherichia coli hemolysin protein across both bacterial membranes. EMBO J., 8:595-605, 1989).

Additional examples of signal sequences include, without limitation, aerolysin, alkaline phosphatase gene (phoA), chitinase, endochitinase, α-hemolysin, MIpB, pullulanase, and Yops. See, e.g., Martinez-Canamero et al., mlpB, a gene encoding a new lipoprotein in Myxococcus xanthus. J. Appl. Microbiol., 92:134-39, 2002; Lloyd et al., Molecular characterization of type III secretion signals via analysis of synthetic N-terminal amino acid sequences. Mol. Microbiol., 43:51-59, 2002; Buttner et al., Functional analysis of HrpF, a putative type III translocon protein from Xanthomonas campestris pv. vesicatoria. J. Bacteriol., 184:2389-98, 2002; Folster et al., The extracellular transport signal of the Vibrio cholerae endochitinase (ChiA) is a structural motif located between amino acids 75 and 555. J. Bacteriol., 184:2225-34, 2002; Tan et al., Engineering a novel secretion signal for cross-host recombinant protein expression. Protein Eng., 15:337-45, 2002; Folders et al., Characterization of Pseudomonas aeruginosa chitinase, a gradually secreted protein. J. Bacteriol., 183:7044-52, 2001; Kononova et al., The primary structure of the N-terminal region of mature alkaline phosphatase is critical for secretion and function of the enzyme. Biochemistry (Mosc.), 65:1075-81, 2000; Ghosh et al., Chitinase secretion by encysting Entamoeba invadens and transfected Entamoeba histolytica trophozoites: localization of secretory vesicles, endoplasmic reticulum, and Golgi apparatus. Infect. Immun., 67:3073-81, 1999; Pugsley et al., Two distinct steps in pullulanase secretion by Escherichia coli K12. Mol. Microbiol., 5:865-73, 1991; Kornacker et al., Molecular characterization of pulA and its product, pullulanase, a secreted enzyme of Klebsiella pneumoniae UNF5023. Mol. Microbiol., 4:73-85, 1990; and Mackman et al., Release of a chimeric protein into the medium from Escherichia coli using the C-terminal secretion signal of haemolysin. EMBO J., 6:2835-41, 1987.

In another embodiment of the present invention, the therapeutic agent is transferred into the target cell after binding of the microorganism to the target cell. Additionally, the therapeutic agent may be an expression plasmid that expresses one or more therapeutically-active agents in the microorganism or in the target cell.

The present invention further provides a method for preventing neoplasia in a subject in need of prevention. The method comprises administering to the subject a preventive composition in an amount effective to prevent the neoplasia. The preventive composition comprises: (a) a microorganism that has, on its cell surface, at least one exogenous molecule that binds to an antigen on the surface of a neoplastic cell in the subject; and (b) a preventive agent.

Data from a number of studies indicate that the anti-tumor effects of microorganism infection are partially mediated through stimulation of the host immune system, resulting in enhanced immune responses to tumor cells. For example, the release of lipopolysaccharide (LPS) endotoxins by Gram negative bacteria (e.g., Salmonella) triggers the host immune system (such as macrophages) to express cytokines, such as tumor necrosis factor (TNF) and interleukin-1 (Sebastiani et al., Host immune response to Salmonella enterica serovar Typhimurium infection in mice derived from wild strains. Infect. Immun., 70:1997-2009, 2002; Lamping et al., LPS-binding protein protects mice from septic shock caused by LPS or gram-negative bacteria. Clin. Invest., 101:2065-71268, 1998; Ramachandra et al., Inhibition of lipid A- and lipopolysaccharide-induced cytokine secretion, B cell mitogenesis, and lethal shock by lipid A-specific murine monoclonal antibodies. J. Infect. Dis., 167:1151-59, 1993). Cytokines, in turn, initiate a cascade of cytokine-mediated reactions which result in the death of tumor cells.

Accordingly, the present invention further provides a method for treating neoplasia in a subject in need of treatment, comprising administering to the subject a therapeutic composition in an amount effective to treat the neoplasia, wherein the therapeutic composition consists of a microorganism that has, on its cell surface, at least one exogenous molecule that binds to an antigen on the surface of a neoplastic cell in the subject. The neoplasia may be any of those described above, but is preferably a colon tumor. Unlike the other therapeutic compositions described above, this therapeutic composition does not comprise a therapeutic agent. The anti-tumor effects of the microorganism of the composition are permitted to work alone to treat the neoplasia. An exogenous polypeptide expressed on the surface of the microorganism may assist in targeting the therapeutic composition to the site of the neoplasm to be treated.

Additionally, the present invention provides a method for preventing neoplasia in a subject in need of prevention, comprising administering to the subject a preventive composition in an amount effective to prevent the neoplasia. The preventive composition consists of a microorganism that has, on its cell surface, at least one exogenous molecule that binds to an antigen on the surface of a neoplastic cell in the subject.

The present invention is described in the following Examples, which are set forth to aid in the understanding of the invention, and should not be construed to limit in any way the scope of the invention as defined in the claims which follow thereafter.

### EXAMPLES

### Example 1

### Generation of an Attenuated Salmonella Typhimurium Vector Displaying Anti-Cea Antibody

A Salmonella typhimurium strain (VNP20009) was used to construct an attenuated vector expressing a high-affinity murine antibody for CEA in its outer membrane. Surface display of a scFv antibody for polypeptide library screening has been achieved in Escherichia coli (E. coli) using Lpp-OmpA fusion (Francisco et al., Production and fluorescence-activated cell sorting of Escherichia coli expressing a functional antibody fragment on the external surface. Proc. Natl. Acad. Sci. USA, 90:10444-48, 1993). This protocol was used in the present study to display CEA-specific scFv on the surface of Salmonella typhimurium VNP20009.

The tripartite fusion construct was based on the MoPac2 plasmid bearing the Ipp-ompA E. coli cell surface targeting and anchoring motif in between the NdeI and SfiI site (Francisco et al., Transport and anchoring of β-lactamase to the external surface of Escherichia coli. Proc. Natl. Acad. Sci. USA, 89:2713-7, 1992). The sequences of anti-CEA diabody T84.66VL-GS8 linker-VH (T84.66-GS8) or scFv T-84.66VL-GS18 linker-VH (T84.66-GS18) were cloned between XbaI and HindIII in pGEM 11zf(−) or pUC18 respectively (Wu et al., Tumor localization of anti-CEA single-chain Fvs: improved targeting by non-covalent dimers. Immunotechnology, 2:21-36, 1996).

For the fusion to Ipp-ompA-scFv, the mammalian leader sequence was removed from cDNA of scFv by PCR, using the following primers: forward primer—CCT AGTCTAGACTAGACATTGTACTGACCCAATC; reverse primer—TTTACTATTACC ATTCGCAG. Purified plasmids containing full-length cDNA for scFv were used as templates for PCR reactions (200 ng of DNA; Hi-Fi Taq polymerase (Invitrogen Corporation, Carlsbad, Calif.); 30 cycles at 94° C. for 30 sec, 55° C. for 30 sec, and 72° C. for 45 sec). PCR products (551 bp for T84.66-GS8 or 581 bp for T84.66-GS18) were purified with Qiagen columns, digested with XbaI/StuI, and cloned in pGEM 11zf(−)/T84.66-GS8 digested with XbaI and StuI and dephosphorylated with shrimp alkaline phosphatase (Roche, Nutley, N.J.). Resulting constructs contained cDNA for scFv (ΔlsT84.66) with a 60-bp truncation (20 amino acid leader sequence). In order to fuse Δls-scFv to Ipp-ompA, coding sequences for Δls-scFv were obtained from appropriate plasmids (T84.66-G8-823 bp and T84.66-G18-853bp), by digesting with XbaI and HindIII, and, after blunt-ending using the Klenow fragment of DNA polymerase I, were inserted into SfiI-digested and T4-polymerase-blunted pMoPac2. The final construct contained a Plac-driven gene consisting of a tripartite of lpp, ompA, and an antibody; a chloramphenicol resistance gene; and a ColEI origin of replication.

Plasmids were introduced into VNP2009 cells by electroporation, and bacteria were grown on LB without NaCl at 37° C. The expression of antibody following IPTG induction (50-500 μM; 25° C. for 6-12 h; Terrific broth) was examined by Western blotting and by flow-cytometric analysis of bacteria stained with goat-anti-mouse-FITC antibodies (Zymed, South San Francisco, Calif.) or CEA-FITC (highly purified CEA protein from Fitzgerald Inc. (Concord, Mass.); FITC-conjugation kit from Sigma (St. Louis, Mo.)). In order to visualize plasmid transfer, through the expression of EGFP reporter protein in mammalian cells infected with VNP20009, an expression cassette containing IE-CMV promoter-EGFP-polyA was inserted into the pMoPac2-scFv plasmid. The cassette was obtained by digesting pcDNA3.1-EGFP with NruI and DraIII. The resulting cassette, containing a 1323-bp fragment, was blunted with T4 DNA polymerase, and ligated into HindIII-digested and Klenow-fragment-of-DNA-polymerase-I-blunted pMoPac2-scFv.

### Example 2

### Construction of a Recombinant Salmonella Vector Displaying Anti-Cea Antibody: A Novel Method for Targeting Colon Adenocarcinomas

An attenuated strain of Salmonella typhimurium (AroA, SL7207) has been used as a vehicle for oral genetic immunization. The mechanism of immunization relies on a natural route of entry through M cells dispersed among epithelial cells lining the gastrointestinal (GI) tract. In addition to inducing apoptosis of infected cells, expression plasmids can be transferred from Salmonella to the nucleus of host APC. The efficiency of plasmid transfer, and the resulting therapeutic effect of any given gene, might be substantially enhanced by directing Salmonella straight into epithelial cells.

Adhesion of a bacterium to its target cell is the first step required for the infection. Carcinoembryonic antigen (CEA), which is a membrane-bound glycoprotein expressed abundantly on epithelial cancerous cells, is an excellent target for bacterial adhesion and subsequent infection. To direct bacterial entry into the epithelial cells, a S. typhimurium strain SL7207 was engineered to express a high-affinity murine antibody for CEA in its outer membrane. Surface expression of the antibodies was achieved by the fusion of F11.39 scFv fragments or T84.66 diabodies with Lpp/OmpA. The final construct contained a Plac-driven gene consisting of a tripartite fusion of: (i) 87 bp from lpp (coding 29 amino acids (aa), including a 9-aa signal sequence from the N-terminal of the mature major E. coli lipoprotein); (ii) 339 bp from ompA (coding for aa 46-159 of the E. coli outer membrane protein); and (iii) 711 bp from F11.39, coding scFv aa 21-237, or 740 bp from T84-66, coding diabody aa 21-247. The predicted structure of the protein consists of 5 membrane-spanning β strands with 2 surface-exposed loops, with antibodies completely exposed on the surface.

The expression of antibodies following IPTG induction was examined by flow-cytometric analysis with goat-anti-mouse-FITC and by Western blot. The efficiency of plasmid transfer (pcDNA3.1zeo(+)/GFP) was evaluated in murine colon tumor cells (MC38) transduced with LXSN/CEA retroviral vector and a human CEA transgenic mouse model.

### Example 3

### Anti-Tumor Effect of the Salmonella Typhimurium S17207 Vector in Murine Tumor Models

The anti-tumor effects of the engineered Salmonella typhimurium SL7207 vectors that express an E. coli cytosine deaminase (CD) may be tested using mice bearing colon adenocarcinomas. For example, SL7207 may be injected intravenously, at doses ranging from 1×10² to 1×10⁹ c.f.u./mouse. 5-fluorocytosine (5-FC) may then be injected intraperitoneally, at doses ranging from 1 μg/kg to 500 mg/kg. Experiments using mice with immune-system deficiencies may demonstrate that the anti-tumor effects of SL7207/5-FC do not depend on the presence of T or B cells. In addition, SL7207, given orally and intravenously, may also inhibit the growth of colon adenocarcinomas in mice.

While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A composition for delivering an agent to a target cell, comprising:
(a) a microorganism that has, on its cell surface, at least one exogenous molecule that binds to an antigen on the surface of a target cell; and
(b) an agent.

2. The composition of claim 1, wherein the microorganism is selected from the group consisting of algae, bacteria, fungi, and protozoa.

3. The composition of claim 2, wherein the microorganism is a bacterium.

4. The composition of claim 3, wherein the bacterium is selected from the group consisting of Escherichia coli, Mycobacterium, Salmonella, and Shigella.

5. The composition of claim 4, wherein the Salmonella is Salmonella typhimurium VNP20009 or Salmonella typhimurium SL7207.

6. The composition of claim 1, wherein the microorganism expresses the exogenous molecule.

7. The composition of claim 6, wherein the microorganism transiently expresses the exogenous molecule.

8. The composition of claim 1, wherein the microorganism is attenuated.

9. The composition of claim 1, wherein the exogenous molecule is a polypeptide or a fragment thereof.

10. The composition of claim 9, wherein the polypeptide is an antibody.

11. The composition of claim 10, wherein the antibody is a mammalian antibody.

12. The composition of claim 11, wherein the antibody is a human antibody.

13. The composition of claim 10, wherein the antibody is a chimeric antibody.

14. The composition of claim 13, wherein the chimeric antibody is a humanized antibody.

15. The composition of claim 10, wherein the antibody is a single-chain antibody.

16. The composition of claim 1, wherein the target cell is a neoplastic cell.

17. The composition of claim 16, wherein the neoplastic cell is a solid-tumor cell.

18. The composition of claim 17, wherein the solid-tumor cell is a colon-tumor cell.

19. The composition of claim 16, wherein the neoplastic cell is a carcinoembryonic-antigen- (CEA)-expressing cell.

20. The composition of claim 19, wherein the CEA-expressing cell is selected from the group consisting of a bowel cancer cell, a breast cancer cell, a cervical cancer cell, a colon cancer cell, an esophageal cancer cell, a head cancer cell, a liver cancer cell, a lung cancer cell, a neck cancer cell, an ovarian cancer cell, a pancreatic cancer cell, and a stomach cancer cell.

21. The composition of claim 20, wherein the CEA-expressing cell is a colon cancer cell.

22. The composition of claim 16, wherein the antigen is a neoplasm-specific antigen.

23. The composition of claim 16, wherein the antigen is selected from the group consisting of CAK1, CDK4, CDR2, carcinoembryonic antigen (CEA), disialoganglioside GD2, HER-2, large external antigen (LEA), MAGEs, MUC1, p21, podocalyxin, Ras, UK114, and WT1.

24. The composition of claim 23, wherein the antigen is a CEA.

25. The composition of claim 1, wherein the agent is selected from the group consisting of a diagnostic agent, a labelling agent, a preventive agent, and a therapeutic agent.

26. The composition of claim 25, wherein the therapeutic agent is selected from the group consisting of an anti-tumor compound, a lipid, a nucleic acid, a polypeptide, a polysaccharide, and a pro-drug.

27. The composition of claim 26, wherein the nucleic acid is a plasmid.

28. The composition of claim 27, wherein the plasmid comprises at least one gene-silencing cassette.

29. The composition of claim 27, wherein the plasmid is an expression plasmid.

30. The composition of claim 25, wherein the polypeptide is selected from the group consisting of an antibody, an anti-proliferation factor, an immuno-enhancing factor, a pro-apoptotic factor, a pro-drug converting enzyme, and any fragment thereof.

31. The composition of claim 30, wherein the polypeptide is modified by glycosylation or lipid linkage.

32. A vaccine comprising:
(a) at least one microorganism that has, on its cell surface, at least one exogenous molecule that binds to an antigen on the surface of a target cell;
(b) an agent; and
(c) a pharmaceutically-acceptable carrier.

33. A method for treating neoplasia in a subject in need of treatment, comprising administering to the subject a therapeutic composition in an amount effective to treat the neoplasia, wherein the therapeutic composition comprises:
(a) a microorganism that has, on its cell surface, at least one exogenous molecule that binds to an antigen on the surface of a neoplastic cell in the subject; and
(b) a therapeutic agent.

34. The method of claim 33, wherein the neoplasia is a solid tumor.

35. The method of claim 34, wherein the solid tumor is a colon tumor.

36. The method of claim 35, wherein the solid tumor expresses carcinoembryonic antigen (CEA).

37. The method of claim 36, wherein the solid tumor is selected from the group consisting of a bowel tumor, a breast tumor, a cervical tumor, a colon tumor, an esophageal tumor, a head tumor, a liver tumor, a lung tumor, a neck tumor, an ovarian tumor, a pancreatic tumor, and a stomach tumor.

38. The method of claim 37, wherein the solid tumor is a colon tumor.

39. The method of claim 33, wherein the microorganism is selected from the group consisting of algae, bacteria, fungi, and protozoa.

40. The method of claim 39, wherein the microorganism is a bacterium.

41. The method of claim 40, wherein the bacterium is selected from the group consisting of Escherichia coli, Mycobacterium, Salmonella, and Shigella.

42. The method of claim 41, wherein the Salmonella is Salmonella typhimurium VNP20009 or Salmonella typhimurium SL7207.

43. The method of claim 33, wherein the microorganism expresses the exogenous molecule.

44. The method of claim 43, wherein the microorganism transiently expresses the exogenous molecule.

45. The method of claim 33, wherein the microorganism is attenuated.

46. The method of claim 33, wherein the exogenous molecule is a polypeptide or a fragment thereof.

47. The method of claim 46, wherein the polypeptide is an antibody.

48. The method of claim 47, wherein the antibody is a mammalian antibody.

49. The method of claim 48, wherein the antibody is a human antibody.

50. The method of claim 47, wherein the antibody is a chimeric antibody.

51. The method of claim 50, wherein the chimeric antibody is a humanized antibody.

52. The method of claim 47, wherein the antibody is a single-chain antibody.

53. The method of claim 33, wherein the antigen is a neoplasm-specific antigen.

54. The method of claim 33, wherein the antigen is selected from the group consisting of CAK1, CDK4, CDR2, carcinoembryonic antigen (CEA), disialoganglioside GD2, HER-2, large external antigen (LEA), MAGEs, MUC1, p21, podocalyxin, Ras, UK114, and WT1.

55. The method of claim 54, wherein the antigen is a CEA.

56. The method of claim 33, wherein the therapeutic agent is selected from the group consisting of an anti-tumor compound, a lipid, a nucleic acid, a polypeptide, a polysaccharide, and a pro-drug.

57. The method of claim 56, wherein the nucleic acid is a plasmid.

58. The method of claim 57, wherein the plasmid comprises at least one gene-silencing cassette.

59. The method of claim 57, wherein the plasmid is an expression plasmid.

60. The method of claim 59, wherein the expression plasmid is transferred into the neoplastic cell.

61. The method of claim 59, wherein the expression plasmid expresses at least one peptide in the neoplastic cell.

62. The method of claim 56, wherein the polypeptide is selected from the group consisting of an antibody, an anti-proliferation factor, an immuno-enhancing factor, a pro-apoptotic factor, a pro-drug converting enzyme, and any fragment thereof.

63. The method of claim 62, wherein the polypeptide is modified by glycosylation or lipid linkage.

64. The method of claim 56, wherein the peptide is secreted into the neoplastic cell.

65. A method for treating neoplasia in a subject in need of treatment, comprising administering to the subject a therapeutic composition in an amount effective to treat the neoplasia, wherein the therapeutic composition consists of a microorganism that has, on its cell surface, at least one exogenous molecule that binds to an antigen on the surface of a neoplastic cell in the subject.
